(19) United States
(12) Patent Application Publication
(10) Pub. No.: US 2006/0234210 A1
(43) Pub. Date: Oct. 19, 2006

(54) Filtration device and method for removing selected materials from biological fluids

(75) Inventors: Daniel J. Kenan, Chapel Hill, NC (US); Dale J. Christensen, Cary, NC (US); Mark W. Grinstaff, Brookline, MA (US)

Correspondence Address: AFFINERGY, INC., P.O. BOX 14650, DURHAM, NC, 27709, US

(73) Assignee: Affinergy, Inc., Research Triangle Park, NC (US)

(21) Appl. No.: 11/105,202

(22) Filed: Apr. 13, 2005

Related U.S. Application Data
(60) Provisional application No. 60/562,166, filed on Apr. 14, 2004.
(60) Provisional application No. 60/590,184, filed on Jul. 22, 2004.

(51) Int. Cl. C12Q 1/70; G01N 33/53

(52) U.S. Cl. 435/5; 422/101; 435/287.1; 436/177; 435/7.1; 210/615; 210/406

(57) ABSTRACT

A filter and method for removing selected materials from a biological fluid sample are provided. The filter comprises an outer housing, inlet, and outlet. A plurality of filter surfaces are provided within the outer housing, and at least one coating is applied to the filter surfaces. The at least one coating comprises at least two binding modules that are in turn selectively bound to one another. One binding module is selectively bound to the filter surfaces and another binding module is configured to bind selectively to the selected materials that are to be removed from the fluid sample. As the fluid sample is allowed to pass through the inlet, outer housing, and outlet, the selected materials are selectively bound to the filter surfaces via the coating, thus producing a filtered product at the outlet.

### CROSS-REFERENCE TO RELATED APPLICATIONS

This application claims the benefit of U.S. Provisional Application No. 60/562,166, filed Apr. 14, 2004, and U.S. Provisional Application No. 60/590,184, filed Jul. 22, 2004, both of which applications are hereby incorporated herein in their entirety by reference.

### FEDERALLY SPONSORED RESEARCH OR DEVELOPMENT

The research underlying this invention was supported in part with funds from NIH grant no. R01 CA77042. The United States Government may have an interest in the subject matter of this invention.

### FIELD OF THE INVENTION

The present invention relates to filter assemblies and methods for removing various materials from biological fluids. The filter assemblies can be used to remove undesirable materials from biological fluids. In other embodiments, the filter assemblies harvest particular cell populations and/or cell products from blood. Methods of the invention utilize the disclosed filter assemblies for selective purification of biological fluids and/or the isolation of particular cells or cell products from biological fluids, including blood.

### BACKGROUND OF THE INVENTION

Many medical treatments require or are improved by the removal of particular materials from a biological fluid. For example, it is advantageous to remove specialized leukocytes known as lymphocytes from donated blood before it is provided to a blood donee. Lymphocytes act to mediate the immune response in the donor's body by distinguishing between foreign matter and “self” matter in the bloodstream. Thus, removal of lymphocytes from donated blood tends to reduce the occurrence of transfusion complications such as “graft versus host” (GVH) disease that results as introduced (donor) T-cells attack the cells already present in the blood donee's circulatory system. Moreover, nucleated cells in blood such as granulocytes and lymphocytes may harbor viral infections such as cytomegalovirus, and removal of nucleated cells reduces risk of post-transfusion infection.

In addition, it is often desirable to isolate cells or other small or rare component from a biological fluid for use in various applications. For example, it is desirable to have the ability to selectively harvest particular components from blood samples such as dendritic cells and stem cells, which may be useful for clinical applications and further study.

Some existing methods of blood purification use filters composed of synthetic materials which operate by way of size exclusion to purify blood. Such methods are disclosed in U.S. Pat. Nos. 5,362,406 to Gsell et al and 5,344,561 to Pall et al. These methods rely on specified pore sizes in a mass of microfibers to retain particles and blood components of a size exceeding the graded pore size. In addition, these filters specify a critical wetting surface tension (CWST) value in order to characterize the wettability of the mass of microfibers.

In addition, some existing leukocyte reduction systems such as those disclosed in U.S. Pat. No. 6,544,751 to Brandwein et al. function by binding leukocytes and other rare cells on a woven polyester filter. The cell collection system in the Brandwein '751 patent operates by allowing the blood to flow through the filter and into a collection bag, leaving the collected cells in the filter system to be discarded or eluted for further use.

Such filters could be improved with a filter coating and filter mechanism utilizing the coating that could selectively filter blood components by chemically binding the blood components (stem cells, for example) to a synthetic filter surface without altering the native molecular structure, properties, and/or functionality of either the synthetic material or the selected blood component. In addition, there exists a need for a coating that is easily applied to and then eluted from existing filter elements such that standard filter elements can be treated with different specialized coatings to filter out selected biological fluid components using the same filter equipment. In addition, there exists a need for a selective filtration system with a selective binding module that is easily applied to a variety of different filter surfaces and filter geometries so that the mechanical aspects of the filtration system (such as filter shear, filter fluid pathways, and filter surface area) can be easily altered without producing negative affects on the ability of the binding modules to effectively retain selected fluid components during the filtration process. Such coatings and filtration systems would find use in a wide array of applications.

### BRIEF SUMMARY OF THE INVENTION

The present invention provides an improved filter assembly and improved method for filtration of selected components from biological fluids, including blood. The filter assembly of the present invention comprises an interfacial biomaterial (IFBM) coating which is composed of at least a first binding module and second binding module that are linked together. The IFBM may be coated onto filter elements to form the improved filter assembly and to improve the filter function of the present invention. The IFBMs of the present invention contain at least two binding modules: a first that may be chosen to selectively bind to a filter surface, and a second module, chemically linked to the first module, that is chosen to selectively bind to the fluid component of interest.

### BRIEF DESCRIPTION OF THE SEVERAL VIEWS OF THE DRAWINGS

The foregoing and other objects or features and advantages of the present invention will be made apparent from the detailed description of the preferred embodiments of the invention and from the following list of drawings which are for illustration purposes and are not necessarily to scale:

FIG. 1 is a schematic view of the IFBM utilized in the filter assembly embodiments of the present invention.

FIG. 2 is a schematic of a filter assembly utilizing IFBM-coated polymer beads according to one embodiment of the present invention.

FIG. 3 is a schematic of a filter assembly of the present invention further comprising a bypass valve.

FIG. 4 is a schematic of a filter assembly of the present invention comprising a plurality of filter surface layers arranged in a column.

FIG. 5 is a schematic of a filter assembly of the present invention comprising IFBM-coated polymer beads contained in a collection bag.

### DETAILED DESCRIPTION OF THE INVENTION

The present inventions now will be described more fully hereinafter with reference to the accompanying drawings in which some but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

The filter assembly of the present invention comprises a coating which is an interfacial biomaterial (IFBM) that comprises at least two distinct binding modules that are centrally linked: one binding module binds to a filter surface, while another binding module binds to a selected biological component. According to one embodiment, these binding modules comprise two separate peptide molecules that bind to a synthetic filter surface and a selected biological component, respectively. In this embodiment, the first binding module peptide of the IFBM is designed to bind to a synthetic material of which the filter is made while the second binding module peptide binds specifically to at least one component or biological material that is to be removed from the fluid. In some embodiments, the IFBM binding module peptides are linked by a central macromolecule to form the IFBM; for example, an IFBM may be a single peptide that comprises multiple binding modules. In this manner, the central macromolecule can be said to “link” the IFBM binding modules, or to be a “linker.” The binding modules typically bind non-covalently to the filter material or biological material. The IFBM selection method and IFBM molecule structure are described in U.S. patent application Ser. No. 10/300,694, filed Nov. 20, 2002 and published on Oct. 2, 2003 as publication number 20030185870, which is herein incorporated by reference.

The term “binds specifically” is intended to mean that the binding module is more likely to bind to a selected material or to a selected component than to another material or component. The term “component” is intended to mean any substance or moiety that can be separated from a fluid, including, for example, cells, biological contaminants such as prions or toxins, or chemicals. Components which can be removed from a biological fluid include cell populations such as macrophages or T-cells and also, for example, malignant cells, prions, viruses, microbes (including bacteria, yeast, and fungi), clotting factors, and compounds such as cholesterol, toxins, and cytokines. Malignant or cancerous cells can be from a liquid tumor such as, for example, leukemia, or can be cells that were shed from a solid tumor. T-cells can be autoreactive T-cells, such as, for example, those found in patients having autoimmune diseases such as multiple sclerosis. A fluid sample which has been exposed to a filter of the invention is a “filtered fluid product.” In some embodiments, the filtered fluid product has a decreased level of at least one component in comparison to the original fluid sample.

By “biological fluid” is intended any fluid which contains at least one biological component. Accordingly, this term encompasses unpurified bodily fluids (such as, for example, whole blood, urine, or pleural fluid), partially purified or separated fluids (such as, for example, plasma), and non-biological fluids which nevertheless contain at least one biological component such as, for example, an isotonic solution to which blood cells have been added. Thus, the filter assemblies of the invention find use in applications such as dialysis to remove waste products from blood and also find use in the treatment of diseases such as cancer or sepsis to remove undesirable malignant cells or bacteria and/or toxins from the blood of a patient. A patient may be a human or a patient may be a non-human mammal or a non-mammal. Thus, biological fluids include, but are not limited to, whole blood, serum, plasma, urine, cerebral spinal fluid (“CSF”), saliva, tears, exudates from wounds, interstitial fluids, semen, ascitic tumor fluid, and breast milk.

The filter surfaces of the invention utilize a polymer base that may comprise woven or non-woven polymeric materials. In other embodiments, the filter elements of the present invention may comprise a container filled with polymeric beads that are, in turn, coated with the IFBMs of the present invention wherein the first binding module comprises a peptide that selectively binds to the surface of the polymeric beads. Materials suitable for constructing filters are known in the art and include, for example, natural polymers such as cellulose and its derivatives or a material such as polyolefin, polyamide, polyimide, polyurethane, polyester, polysulfone, polyacrylonitrile, polyethersulfone, poly(meth)acrylate, butadiene-acrylonitrile copolymer, ethylene-vinylalcohol copolymer and polyvinylacetal, or a mixture of any of these. Because the binding elements of the invention are suitable for binding to such materials, they also find use in other applications involving such materials.

The IFBM coating of the present invention can be used with a variety of filter configurations. For example, the filter surfaces of this invention utilize a polymer base that may comprise woven or non-woven polymeric materials. In addition, the filter surface materials may be formed into various filter geometries to suit the specific type of clinical or research application. In one embodiment of the filter assembly, the filter elements comprise a pleated membrane device or a column comprising an IFBM-coated polymer matrix. In other embodiments, the filter elements may consist of polymer tubing through which the fluid passes and to which the IFBMs of the present invention are bound. In other embodiments, the filter assembly may further comprise a bypass valve such that the collected fluid components can be eluted from the filter surfaces and collected for further study and/or culture.

The improved method of filtering fluid components of the present invention consists of the following steps: (1) providing an IFBM-coated filter assembly wherein at least one binding module of the IFBM binds specifically to the fluid component that is being filtered, (2) passing a fluid solution sample through the IFBM-coated filter assembly, (3) collecting the filtered fluid solution sample downstream of the IFBM-coated filter assembly. According to other advantageous embodiments, the filter method of the present invention may further comprise an eluting step wherein the filtered fluid components are eluted from the coated filter surface for collection and further downstream processing, study, or culture.

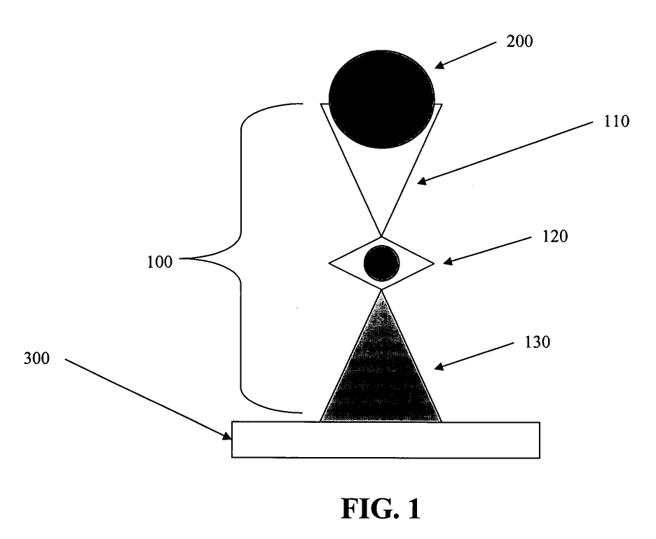

Referring to FIG. 1, a schematic of an IFBM used in the fluid filter assemblies of the present invention is shown. According to the embodiment shown, the IFBM 100 comprises three main components: a first binding module 130 that binds specifically to the filter surface 300, a second binding module 110 that binds specifically to a specific fluid component 200, and a “linker” or linking macromolecule 120 that acts to bind the first binding module 130 to the second binding module 110.

According to alternate embodiments, the IFBM may contain various binding modules that are known to specifically bind with a variety of filter materials and a variety of specific components that are to be removed or harvested from a fluid sample. In addition, according to the method embodiments of the invention, the IFBM may be eluted from the filter surface and subsequently, another IFBM with a different binding module may be applied to the filter surfaces so that the filter is then capable of retaining another specific fluid component. In yet another embodiment, binding modules may be directly bonded to each other without the use of a linking macromolecule 120; for example, a single peptide may comprise more than one binding module.

Thus, IFBMs comprise at least a first binding module and a second binding module which are attached to each other directly or via a linker. To create an IFBM of the invention, a binding module is identified which binds to a particular filter material, and another binding module is identified which binds to a component or a material which is to be removed from the fluid. These binding modules are then combined to create an IFBM. The modules may be combined using recombinant DNA technologies or may be combined using a linker to attach the modules to each other.

Binding modules may be peptides, antibodies or antibody fragments, small molecule ligands, polynucleotides, oligonucleotides, complexes comprising any of these, or various molecules and/or compounds. For example, in some embodiments, a binding module is a transcription complex or a protein capable of binding to penicillin. Binding modules which are peptides may be identified as described in pending U.S. patent application Ser. No. 10/300,694, filed Nov. 20, 2002 and published on Oct. 2, 2003 as publication number 20030185870. In some embodiments, binding modules may be identified by screening phage display libraries for binding to filter materials such as nylon or for binding to a particular fluid component, for example, for binding to populations of particular blood cells.

The term “antibody” as used herein with reference to a binding module encompasses single chain antibodies. Thus, an antibody useful as a binding module may be a single chain variable fragment antibody (scFv). A single chain antibody is an antibody comprising a variable heavy and a variable light chain that are joined together, either directly or via a peptide linker, to form a continuous polypeptide. The term “single chain antibody” as used herein encompasses an immunoglobulin protein or a functional portion thereof, including but not limited to a monoclonal antibody, a chimeric antibody, a hybrid antibody, a mutagenized antibody, a humanized antibody, and antibody fragments that comprise an antigen binding site (e.g., Fab and Fv antibody fragments).

Phage display technology is well-known in the art. Using phage display, a library of diverse peptides can be presented to a target substrate, and peptides that specifically bind to the substrate can be selected for use as binding modules. Multiple serial rounds of selection, called “panning,” may be used. As is known in the art, any one of a variety of libraries and panning methods can be employed to identify a binding module that is useful in the methods of the invention. For example, libraries of antibodies or antibody fragments may be used to identify antibodies or fragments that bind to particular cell populations or to viruses (see, e.g., U.S. Pat. Nos. 6,174,708, 6,057,098, 5,922,254, 5,840,479, 5,780,225, 5,702,892, and 5,667,988). Panning methods can include, for example, solution phase screening, solid phase screening, or cell-based screening. Once a candidate binding module is identified, directed or random mutagenesis of the sequence may be used to optimize the binding properties of the binding module.

A library can comprise a random collection of molecules. Alternatively, a library can comprise a collection of molecules having a bias for a particular sequence, structure, or conformation. See, e.g., U.S. Pat. Nos. 5,264,563 and 5,824,483. Methods for preparing libraries containing diverse populations of various types of molecules are known in the art, and numerous libraries are also commercially available. Methods for preparing phage libraries can be found, for example, in Kay et al. (1996) Phage Display of Peptides and Proteins (San Diego, Academic Press); Barbas (2001) Phage Display: A Laboratory Manual (Cold Spring Harbor Laboratory Press, Cold Spring Harbor, N.Y.)

A binding module that is a peptide comprises at least or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 200, or up to 300 amino acids. Peptides useful as a binding module can be linear, branched, or cyclic, and can include non-peptidyl moieties. The term “peptide” broadly refers to an amino acid chain that includes naturally occurring amino acids, synthetic amino acids, genetically encoded amino acids, non-genetically encoded amino acids, and combinations thereof. Peptides can include both L-form and D-form amino acids.

A peptide of the present invention can be subject to various changes, substitutions, insertions, and deletions where such changes provide for certain advantages in its use. Thus, the term “peptide” encompasses any of a variety of forms of peptide derivatives including, for example, amides, conjugates with proteins, cyclone peptides, polymerized peptides, conservatively substituted variants, analogs, fragments, chemically modified peptides, and peptide mimetics. Any peptide that has desired binding characteristics can be used in the practice of the present invention.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine.

The term “conservatively substituted variant” refers to a peptide having an amino acid residue sequence substantially identical to a sequence of a reference peptide in which one or more residues have been conservatively substituted with a functionally similar residue such that the conservatively substituted variant will bind to the same binding partner with substantially the same affinity as the parental variant and will prevent binding of the parental variant in a competition assay. In one embodiment, a conservatively substituted variant displays a similar binding specificity when compared to the reference peptide. The phrase “conservatively substituted variant” also includes peptides wherein a residue is replaced with a chemically derivatized residue.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue such as aspartic acid or glutamic acid for another.

Peptides which are binding modules of the present invention also include peptides having one or more substitutions, additions and/or deletions of residues relative to the sequence of an exemplary peptide sequence is disclosed herein, so long as the requisite binding properties are retained. Thus, binding modules of the invention include peptides that differ from the exemplary sequences disclosed herein by 1, 2, 3, 4, 5, 6, 7, 8, 9, 10, 11, 12, 13, 14, 15, 16, 17, 18, 19, or 20 amino acids. That is, binding modules of the invention include peptides that share sequence identity with the exemplary sequences disclosed herein of at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater sequence identity. Sequence identity may be calculated manually or it may be calculated using a computer implementation of a mathematical algorithm, for example, GAP, BESTFIT, BLAST, FASTA, and TFASTA in the Wisconsin Genetics Software Package of Genetics Computer Group, Version 10 (available from Accelrys, 9685 Scranton Road, San Diego, Calif., 92121, USA). The scoring matrix used in Version 10 of the Wisconsin Genetics Software Package is BLOSUM62 (see Henikoff and Henikoff (1989) Proc. Natl. Acad. Sci. USA 89: 10915). Alignments using these programs can be performed using the default parameters.

Unless otherwise specified, alignments are performed using the BLAST program with default parameters. The BLAST programs of Altschul et al (1990) J. Mol. Biol. 215:403 are based on the algorithm of Karlin and Altschul (1990) Proc. Nat'l. Acad. Sci. USA 87:2264-2268. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) NucleicAcids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10. GAP uses the algorithm of Needleman and Wunsch (1970) J. Mol. Biol. 48:443-453, to find the alignment of two complete sequences that maximizes the number of matches and minimizes the number of gaps.

The invention also provides computers, computer-readable media and integrated systems, including databases that are composed of sequence records including character strings corresponding to SEQ ID NOs: 1-462. Such integrated systems optionally include one or more instruction set for selecting, aligning, translating, reverse-translating or viewing any one or more character strings corresponding to SEQ ID NOs: 1-462 with each other and/or with any additional nucleic acid or amino acid sequence.

The invention additionally provides nucleotide sequences encoding the peptides of the invention.

A peptide can be modified, for example, by terminal-NH₂ acylation (e.g., acetylation, or thioglycolic acid amidation) or by terminal-carboxylamidation (e.g., with ammonia or methylamine). Terminal modifications are useful to reduce susceptibility by proteinase digestion, and to therefore prolong a half-life of peptides in solutions, particularly in biological fluids where proteases can be present.

Peptide cyclization is also a useful modification because of the stable structures formed by cyclization and in view of the biological activities observed for such cyclic peptides. Methods for cyclizing peptides are described, for example, by Schneider & Eberle (1993) Peptides. 1992: Proceedings of the Twenty-Second European Peptide Symposium. Sep. 13-19. 1992. Interlaken, Switzerland, Escom, Leiden, The Netherlands.

Optionally, a binding module peptide can comprise one or more amino acids that have been modified to contain one or more halogens, such as fluorine, bromine, or iodine, to facilitate linking to a linker molecule. As used herein, the term “peptide” also encompasses a peptide wherein one or more of the peptide bonds are replaced by pseudopeptide bonds including but not limited to a carba bond (CH₂—CH₂), a depsi bond (CO—O), a hydroxyethylene bond (CHOH—CH₂), a ketomethylene bond (CO—CH₂), a methylene-oxy bond (CH₂—O), a reduced bond (CH₂—NH), a thiomethylene bond (CH₂—S), an N-modified bond (—NRCO—), and a thiopeptide bond (CS—NH). See e.g., Garbay-Jaureguiberry et al. (1992) Int. J. Pept. Protein Res. 39: 523-527; Tung et al. (1992) Pept. Res. 5: 115-118; Urge et al. (1992) Carbohydr. Res. 235: 83-93; Corringer et al. (1993) J. Med. Chem. 36: 166-172; Pavone et al. (1993) Int. J. Pept. Protein Res. 41: 15-20.

Representative peptides that specifically bind to a non-biological substrate which is a filter material are set forth as SEQ ID NOs: 1-462. Peptide ligands that specifically bind a biological substrate include peptides known in the art to have particular binding specificities. See, for example, Christian et al. (2003) J. Cell Biol. 163: 871-878; Assa-Munt et al. (2001) Biochemistry 40: 2373-2378; Manke et al. (2003) Science 302: 636-639, Samoylova et al. (2003) Mol. Cancer Ther. 11: 1129-1137; Oyama et al. (2003) Cancer Lett. 202: 219-230; Liu et al. (2004) Int. J. Cancer 109: 49-57; Rasmussen et al. (2002) Cancer Gene Ther. 9: 606-612; and references cited therein. While exemplary peptide sequences are disclosed herein, one of skill will appreciate that the binding properties conferred by those sequences may be attributable to only some of the amino acids comprised by the sequences. Thus, a sequence which comprises only a portion of an exemplary sequence disclosed herein may have substantially the same binding properties as the full-length exemplary sequence. Thus, also useful as binding modules are sequences that comprise only 4, 5, 6, 7, 8, 9, 10, 11, 12, 13, 14, 15, 16, 17, or 18 of the amino acids in a particular exemplary sequence, and such amino acids may be contiguous or non-contiguous in the exemplary sequence. Such amino acids may be concentrated at the amino-terminal end of the exemplary peptide (for example, 4 amino acids maybe concentrated in the first 5, 6, 7, 8, 9, 10, 11, 12, 13, 14, 15, 16, 17, or 18 amino acids of the peptide) or they may be dispersed throughout the exemplary peptide.

Binding modules of the present invention that are peptides can be synthesized by any of the techniques that are known to those skilled in the art of peptide synthesis. Representative techniques can be found, for example, in Stewart & Young (1969) Solid Phase Peptide Synthesis, (Freeman, San Francisco, Calif.); Merrifield (1969) Adv Enzymol Relat Areas Mol Biol 32:221-296; Fields & Noble (1990) Int J Pept Protein Res 35:161-214; and Bodanszky (1993) Principles of Peptide Synthesis, 2nd Rev. Ed. (Springer-Verlag, Berlin). Representative solid phase synthesis techniques can be found in Andersson et al. (2000) Biopolymers 55: 227-250, references cited therein, and in U.S. Pat. Nos. 6,015,561; 6,015,881; 6,031,071; and 4,244,946. Peptide synthesis in solution is described in Schröder & Lübke (1965) The Peptides (Academic Press, New York, N.Y.). Appropriate protective groups useful for peptide synthesis are described in the above texts and in McOmie (1973) Protective Groups in Organic Chemistry (Plenum Press, London). Peptides, including peptides comprising non-genetically encoded amino acids, can also be produced in a cell-free translation system, such as the system described by Shimizu et al. (2001) Nat Biotechnol 19: 751-755. In addition, peptides having a specified amino acid sequence can be purchased from commercial sources (e.g., Biopeptide Co., LLC of San Diego, Calif.), and PeptidoGenics of Livermore, Calif.).

In some embodiments, a binding domain of the invention can further comprise one or more crosslinking moieties, such as a photocrosslinkable moiety, an ionically crosslinkable moiety, or terminally crosslinkable moiety. The crosslinking moieties can be used to create a two-dimensional or three-dimensional interfacial biomaterial.

The IFBMs of the invention optionally further comprise a linker between the first binding domain and the second binding domain. The linker can facilitate combination of two or more first or second binding domains. In addition, the linker can perform a spacer function to minimize potential steric hindrance between the two or more binding domains. In some embodiments, the linker is substantially biologically inert except for its linking and/or spacer properties.

Suitable linkers comprise one or more straight or branched chain(s) of 2 carbon atoms to about 50 carbon atoms, wherein the chain is fully saturated, fully unsaturated, or a combination thereof. Typically, a linker comprises between 2 and about 100 sites for ligand attachment. The methods employed for linking will vary according to the chemical nature of the binding domains, filter material, and component of interest. One of skill in the art is capable of selecting a suitable linker for a particular IFBM and method of use.

Suitable reactive groups of a linker include but are not limited to amines, carboxylic acids, alcohols, aldehydes, and thiols. An amine group in a linker can form a covalent bond with a carboxylic acid group of a ligand, such as a carboxyl terminus of a peptide ligand. A carboxylic acid group or an aldehyde in a linker can form a covalent bond with the amino terminus of a peptide ligand or other ligand amine group. An alcohol group in a linker can form a covalent bond with the carboxyl terminus of a peptide ligand or other ligand carboxylic acid group. A thiol group in a linker can form a disulfide bond with a cysteine in a peptide ligand or a ligand thiol group.

Additional reactive groups that can be used for linking reactions include, but are not limited to a phosphate, a sulphate, a hydroxide, —SeH, an ester, a silane, urea, urethane, a thiol-urethane, a carbonate, a thio-ether, a thio-ester, a sulfate, an ether, or a combination thereof.

In some embodiments of the invention, a linker comprises a peptide which comprises between 1 and 40 amino acids. Sites for ligand attachment to a peptide ligand include functional groups of the amino acid side chains and the amino and carboxyl terminal groups. Representative peptide linkers with multiple reactive sites include polylysines, polyornithines, polycysteines, polyglutamic acid and polyaspartic acid. Alternatively, substantially inert peptide linkers comprise at least one of polyglycine, polyserine, polyproline, polyalanine, and other oligopeptides comprise at least one of alanyl, serinyl, prolinyl, or glycinyl amino acid residues.

Peptide linkers can be pennant or cascading. The term “pennant polypeptide” refers to a linear peptide. As with polypeptides typically found in nature, the amide bonds of a pennant polypeptide are formed between the terminal amine of one amino acid residue and the terminal carboxylic acid of the next amino acid residue. The term “cascading polypeptide” refers to a branched peptide, wherein at least some of the amide bonds are formed between the side chain functional group of one amino acid residue and the amino terminal group or carboxyl terminal group of the next amino acid residue.

In another embodiment of the invention, a linker can comprise a polymer, including a synthetic polymer or a natural polymer. Representative synthetic polymers include but are not limited to polyethers (e.g., polyethylene glycol; PEG), polyesters (e.g., polylactic acid (PLA) and polyglycolic acid (PGA)), polyamines (e.g., nylon), polyamines (e.g., polymethylmethacrylate; PMMA), polyacrylic acids, polyurethanes, polystyrenes, and other synthetic polymers having a molecular weight of about 200 daltons to about 1000 kilodaltons. Representative natural polymers include but are not limited to hyaluronic acid, alginate, chondroitin sulfate, fibrinogen, fibronectin, albumin, collagen, and other natural polymers having a molecular weight of about 200 daltons to about 20,000 kilodaltons. Polymeric linkers can comprise a diblock polymer, a multi-block copolymer, a comb polymer, a star polymer, a dendritic polymer, a hybrid linear-dendritic polymer, or a random copolymer.

A linker can also comprise a mercapto(amido)carboxylic acid, an acrylamidocarboxylic acid, an acrlyamido-amidotriethylene glycolic acid, and derivatives thereof. See, for example, U.S. Pat. No. 6,280,760. A linker may also comprise a plurality of one or more small flexible amino acids, including, but not limited to, glycine, serine, and threonine.

Methods for linking a linker molecule to a ligand or to a non-binding domain will vary according to the reactive groups present on each molecule. Protocols for linking using the above-mentioned reactive groups and molecules are known to one of skill in the art. See, e.g., Goldman et al. (1997) Cancer Res. 57: 1447-1451; Cheng (1996) Hum. Gene Therapy 7: 275-282; Neri et al. (1997) Nat. Biotechnol. 19: 958-961; Nabel (1997) Current Protocols in Human Genetics, vol. on CD-ROM (John Wiley & Sons, New York); Park et al. (1997) Adv. Pharmacol. 40: 399-435; Pasqualini et al. (1997) Nat. Biotechnol. 15: 542-546; Bauminger & Wilchek (1980) Meth. Enzymol. 70: 151-159; U.S. Pat. Nos. 6,280,760 and 6,071,890; and European Patent Nos. 0 439 095 and 0 712 621. Thus, a linker can be chosen which will permit the dissociation of the IFBM into its component binding modules when certain treatments are applied. In such embodiments, the dissociation of the IFBM may or may not be accompanied by the release of a bound component from the second binding module.

Antibodies for use as binding modules can be identified by panning methods. Alternatively, known antibodies having a desired binding specificity or a desired non-binding quality can be used. For example, U.S. Pat. No. 5,874,542 to Rockwell et al. discloses single chain antibodies that specifically bind to vascular endothelial growth factor (VEGF) receptor. VEGF is expressed, for example, in macrophages (Brown et al. (1992) J. Exp. Med. 176: 1375-1379). Thus, antibodies which bind to cell surface markers can be readily identified by those of skill in the art for use as binding modules, and some cell-specific antibodies are commercially available.

Cell surface markers specific to various cell populations are known in the art. See, e.g., Bleesing & Fleisher (2001) Semin. Hematol. 38: 100-110 (discussing “immunophenotyping”); Chan et al. (2003) Immunol. Lett. 85: 159-163 (discussing NKT cell subsets in infection and inflammation); Kuchroo et al. (2003) Nat. Rev. Immunol. 3: 454-462 (discussing the TIM gene family, some of which are differentially expressed by T_{H}1 and T_{H}2 cells). In addition, methods for identifying cell surface markers are known in the art. See, e.g., Brown (2000) Curr. Opin. Chem. Biol. 4: 16-21. Peptides may be also identified which bind to particular cell types and are therefore useful as binding modules. See, e.g., Rasmussen et al. (2002) Cancer Gene Ther. 9: 606-612 (discussing tumor cell targeting by phage-displayed peptides, including the peptide HEWSYLAPYPWF).

Filter surfaces are coated, for example, by dipping or spraying the IFBM onto the filter material. The coating may be stabilized, for example, by air drying or by lyophilization. However, these treatments are not exclusive, and other coating and stabilization methods may be employed. It will be understood by those of skill in the art that an IFBM coating of the invention may also be used with at least one other filter coating so long as the IFBM coating is still capable of binding to the selected component.

Referring again to FIG. 1, a binding module 110 of the IFBM 100 of the present invention may be tailored to form a peptide bond with a variety of materials that may be found in a blood solution.

It will be understood that IFBMs can be made in order to selectively bind any component for which a binding module can be produced. For example, an IFBM can comprise a first binding module which binds to a filter material and a second binding module which binds to a particular cell population such as, for example, leukocytes. Any component for which a binding module may be made can be removed from the biological fluid. Where desirable, these components can then be removed from the filter for further use. For example, cells may be eluted from the filter in a suitable elution buffer as described in U.S. Pat. No. 6,544,751. In order to remove more than one component during one pass of the fluid through the filter apparatus, filter apparatuses may contain more than one type of filter and/or filters having different coatings. These different filters and any components adhering to them or removed by them can then be treated separately as desired. It will be understood that the filters of the invention may be useful in diagnosis of diseases which are indicated by the presence, absence, or alteration of particular components or cell types.

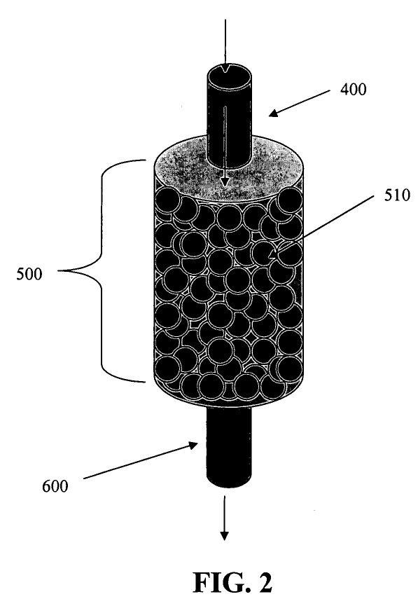

A filter assembly according to one advantageous embodiment of the present invention is shown in FIG. 2. In this filter assembly embodiment, an IFBM 100, as shown, for example, in FIG. 1, is applied to the surfaces of a plurality of polymeric beads 510. The polymeric beads 510 are, in turn, contained within a filter assembly 500 having a cylindrical shape. The filter assembly 500 is provided with an inlet 400 and outlet 600 so that fluid samples may enter the filter assembly through the inlet, interact with the IFBM-coated polymeric beads in the filter assembly, and then pass through the outlet. The inlet, outlet, and polymeric beads of this embodiment are sized so that the polymeric beads are retained within the filter assembly even as the sample passes through the filter assembly. As the sample passes through the filter assembly 500, the IFBM's 100 interact with the sample so that specific components 200 that bind to the binding module 110 of the IFBM are bound to the IFBM's applied to the polymeric beads via peptide bond interactions between the polymeric bead surface 300 and the binding module 130 of the IFBM's 100.

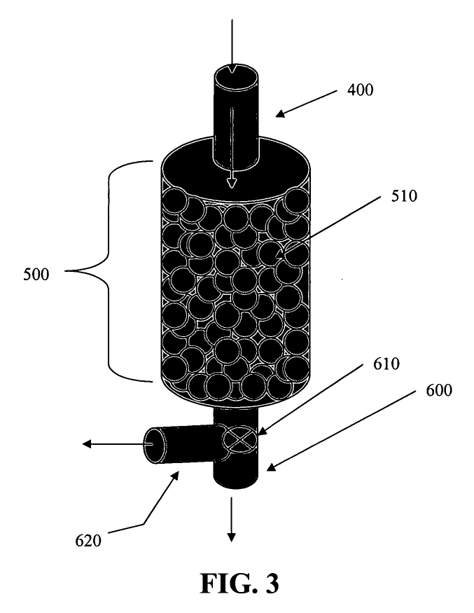

Another advantageous embodiment of the filter assembly of the present invention is shown in FIG. 3. In this embodiment, the filter assembly outlet 600, further comprises a downstream valve 610 and bypass 620 so that the fluids emerging from the filter assembly 500 can be re-routed through the bypass 620. The valve and bypass arrangement of this embodiment allows the user of the filter assembly to elute the captured components 200 from the IFBMs 100 by passing a suitable eluting compound through the filter assembly after the sample has passed through the filter. The eluting compound then acts to detach the captured components from the first binding modules 110 of the IFBM's 100, producing an eluate that passes through the bypass 620 and into a separate collection apparatus. The collection apparatus may comprise, for example, a sterile specimen bag, sterile vial, or any collection vessel suitable for retaining the fluid eluate that emerges from the bypass. Using this embodiment of the filter assembly, the components 200 trapped and subsequently eluted from the IFBM filter apparatus can be collected and preserved for study, replication, and/or culture. In some embodiments, the internal surfaces of the valve 610 may be coated with IFBMs so as to ensure that no residual selected components 200 are allowed to drip through the valve and into the outlet 600.

The valve 610 used in the various embodiments of this invention may be a hand-actuated bypass valve, a solenoid valve which is responsive to an electrical input, or for example, a pneumatically or hydraulically-actuated bypass valve.

In addition, the filter assembly embodiment depicted in FIG. 3 can be used to elute the IFBM's from the polymeric beads 510 held within the filter assembly. The polymeric beads 510, inlet 400, and outlet 600 of this embodiment of the filter assembly may be sized so that as the samples, and/or eluant is passed through the filter assembly, the polymeric beads are retained within the filter assembly and are too large to pass through the outlet. Thus, a suitable eluant may be passed through the filter assembly so as to break the peptide bonds between the polymeric bead filter surface 300 and the binding module 130 of the IFBM 100. The valve 610 may be actuated so that the eluate containing the loose IFBMs 100 are washed through the bypass and thus out of the filter assembly.

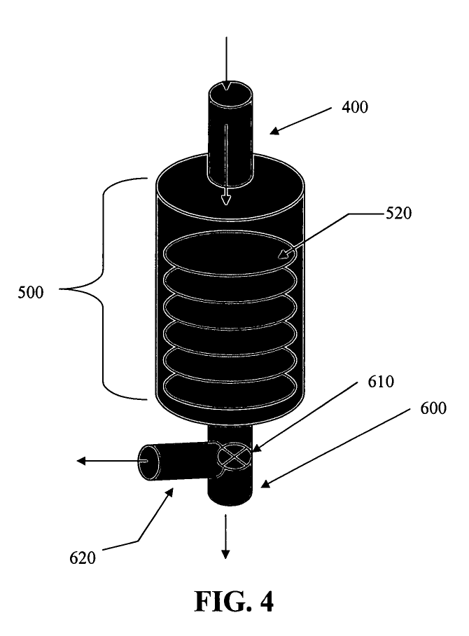

FIG. 4 shows another embodiment of a filter assembly of the present invention wherein the IFBMs are applied to stacked filter layers 520 arranged in a column defined by the filter assembly 500. In this embodiment, the sample flows through the column from the inlet 400 through the stacked filter layers 520, which are coated with the IFBMs 100 of the present invention. The sample then exits the filter assembly through the outlet 600 leaving the selected components 200 retained in the stacked filter layers, via peptide bonds formed with the binding modules 110 of the IFBMs 100. In a manner similar to that described in relation to the embodiment of FIG. 3, the stacked filter layers 520 of this embodiment may be washed with an eluate suitable for breaking the peptide bond between either: (1) the binding module 110 and the selected component 200 or (2) the binding module 130 and the filter surface 300 of the stacked filter layers 520. Thus the selected components 200 bound by the IFBMs can be collected and selectively removed from the filter via a bypass 620 that shunts the eluate flow away from the outlet 600 and towards a suitable collection container.

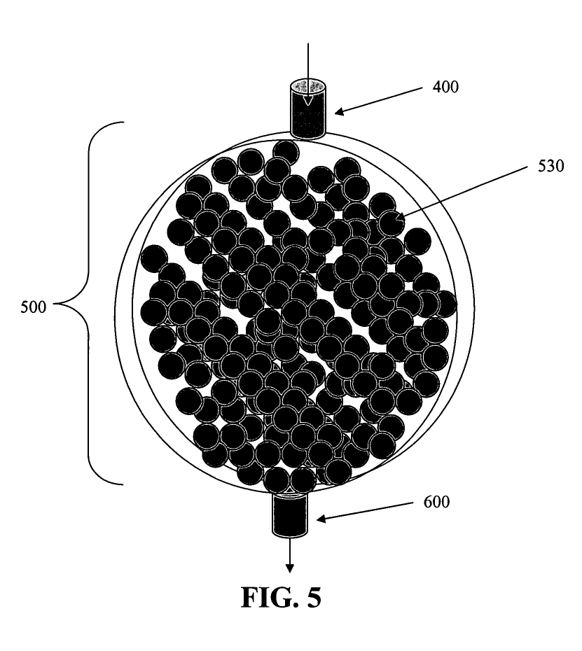

FIG. 5 shows another advantageous embodiment of the filter assembly of the present invention wherein the IFBMs are applied to polymer beads contained within a polymer collection bag that serves as the filter assembly 500. In this embodiment, the sample fluid flows through the collection bag from the inlet 400 through the plurality of polymer beads 530, which are coated with the IFBMs 100 of the present invention. The fluid then exits the collection bag through the outlet 600 leaving the selected components 200 retained in the stacked filter layers, via peptide bonds formed with the binding modules 110 of the IFBMs 100. The inlet and outlet of the collection bag have a diameter smaller than that of the polymer beads so that the polymer beads are retained in the collection bag as the blood sample passes through the filter assembly.

According to other embodiments of the filter assembly of the present invention, filter surfaces 300 may be made of strands of material laid down in a random fashion or may be made of foam or other nonwoven material. Filter surfaces may also be made of woven material. In some embodiments, the filtration assembly 500 may comprise a pleated membrane device. It will be understood that with the coatings of the present invention, it will be desirable in many advantageous embodiments to maximize the surface area of the filter surfaces which can be coated with the IFBMs of the present invention. In addition, the filter assembly may also include tubing, and the tubing may also be coated with IFBMs. For instance, in the embodiment of the filter assembly shown in FIGS. 2, 3, and 4, the inlet 400 and outlet 600 may comprise polymeric tubing that is further coated with IFBMs to more completely filter the blood samples passing therethrough.

The filter surfaces 300 of the present invention may comprise a number of different materials since the binding module 130 of the IFBM 100 may be chosen such that it forms a peptide bond with the specific filter surface 300 chosen for the filter assembly. For example, advantageous embodiments of the present invention may utilize filter surface 300 materials including, polypropylene, polyester, nylon (polyamide), cellulose, regenerated cellulose, polysulphone, PVPF (polyvinylpolyfluoride), and Gore-Tex. In addition, in embodiments using polymer beads 510, polystyrene beads may be used. Polymer beads may also be made from, for example, polypropylene and polyester.

The filter surfaces 300 of the present invention may be further prepared or treated in some embodiments so that the filter surfaces have an improved affinity for the binding module 130 of the IFBM 100. For example, the hydrophobic or hydrophilic properties of the filter surfaces may be altered to improve the affinity capture of any molecule or entity to which an IFBM component can bind. Thus, in some embodiments, the wettability of a filter is enhanced. For example, the wettability of a Teflon filter may be increased by increasing the surface area of the filter surface. The shear, or relative velocities of adjacent layers of fluid flow, produced by a filter may also be altered by adjusting the surface area, wettability, and binding properties of the filter surface using the techniques of the present invention.

Referring again to FIGS. 1 and 2, one embodiment of a method for removing selected blood components 200 from a blood sample may comprise the following steps: (1) providing a filter assembly 500 having an inlet 400, outlet 600, and a plurality of filter surfaces 300 located in the filter assembly, (2) providing an plurality of IFBMs 100 that bind to the filter surfaces via a binding module 130 and comprise a binding module 110 that binds to the selected component 200, (3) filtering a sample through the filter assembly so that the selected component interacts with the second binding module and is bound thereto while the remaining sample exits the filter assembly via an outlet 600.

An alternate method embodiment of the present invention provides a method for selectively harvesting selected components using a filter assembly such as that which is shown in FIG. 3. According to this embodiment, the method further comprises the steps of: (1) sealing the outlet 600 of the filter assembly and opening a bypass 620 via the actuation of a valve 620, (2) washing the filter surfaces with an eluting compound suitable for breaking the peptide bonds formed between the binding module 110 and the selected component 200 such that an eluate is formed containing the eluting compound and the selected component, and (3) collecting the eluate in a collection apparatus via the bypass 620.

The method embodiments described herein can be modified, for example, for use in the following applications. IFBM coated filters may be used during the fluid collection process. In some embodiments, the IFBM-coated filter or filters may be used in a blood collection apparatus between a needle which is inserted into the patient's body and a collection bag attached to the outlet 600 of the filter assembly. Filter assemblies according to the present invention may also be used as devices to treat the fluid flowing through them, for example, by performing a biocide function to remove selected biological materials from a blood sample. The filter assemblies may also be used in dialysis in order to remove selected contaminants and waste products from a blood sample; in such embodiments, the filter assembly may be coated with IFBMs that comprise second binding modules that are adapted to form peptide bonds with the respective selected blood contaminants (e.g., waste products).

Furthermore, the filter assemblies shown in FIGS. 2, 3, 4, and 5 may be used in series to collect a plurality of different components as a fluid sample is passed through the filter assemblies having different IFBM 100 coatings attached to their respective filter surfaces 300. In addition, more than one type of IFBM 100 may be applied to the plurality of filter surfaces located within a single filter assembly so that more than one selected component may be removed from a fluid sample as it passes through a single filter assembly. In addition, in other embodiments, filter assemblies may therefore contain more than one type of filter surface and/or filters having different IFBM coatings in order to remove more than one component during one pass of the fluid through the filter apparatus. These different filters and any selected components adhering to them or removed by them can then be treated separately.

While the methods described herein are claimed by reference to particular steps, one of skill will appreciate that in some instances the order of the method steps may be varied so long as the object of the invention is achieved, i.e., providing a method which will accomplish the stated purposes.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A filter for removing at least one selected component from a fluid sample that passes through the filter, comprising:
a) an inlet;
b) an outlet;
c) a filter assembly comprising an outer housing and a plurality of filter surfaces located and contained within the outer housing; and
d) a coating bound to the at least one filter surface, wherein said coating further comprises:
i) a first binding module, configured to selectively bind to the plurality of filter surfaces, and
ii) a second binding module, configured to selectively bind to the first binding module and to the at least one selected fluid component such that the at least one selected fluid component is selectively bound to the plurality of filter surfaces via the coating and a filtered fluid product emerges from the outlet into the collection container.

2. The filter of claim 1, wherein the coating further comprises a central macromolecule configured to bind the first binding module to the second binding module.

3. The filter of claim 1, wherein the first binding module is linked to the second binding module by a peptide bond.

4. The filter of claim 1, wherein the plurality of filter surfaces comprise a polymer.

5. The filter of claim 1, wherein the outer housing comprises a polymer cylinder and the plurality of filter surfaces are located on a plurality of polymeric beads held inside the polymer cylinder.

6. The filter of claim 1, wherein the outlet further comprises a valve and a bypass such that the valve may be actuated to close the outlet and open the bypass.

7. The filter of claim 6, further comprising an eluate container attached to the bypass via a fluid-tight conduit.

8. The filter of claim 1, further comprising a collection container connected to the outlet via a fluid-tight conduit.

9. The filter of claim 1, wherein the outer housing comprises a polymer cylinder and the plurality of filter surfaces are located on a plurality of vertically-stacked polymeric layers held inside the polymer cylinder.

10. The filter of claim 1, wherein the plurality of filter surfaces comprise woven polymeric material.

11. The filter of claim 1, wherein the plurality of filter surfaces comprise a pleated polymer sheet.

12. The filter of claim 1, wherein the outer housing comprises a flexible polymer bag and the plurality of filter surfaces are located on a plurality of polymeric beads held inside the flexible polymer bag.

13. The filter of claim 1, wherein said fluid sample is a blood sample and wherein said at least one selected fluid component is a blood component chosen from the group consisting of:
a) leukocytes;
b) granulocytes;
c) lymphocytes;
d) dendritic cells;
e) stem cells;
f) platelets;
g) malignant cells;
h) proteins;
i) prions;
j) antibodies;
k) growth factors;
l) cytokines;
m) hormones;
n) lipids;
o) cholesterol;
p) toxins;
q) bacteria;
r) yeasts;
s) fungi;
t) viruses; and
u) protozoan parasites.

14. The filter of claim 1, wherein the outer housing comprises a section of hollow polymer tubing defining an inner surface and the plurality of filter surfaces are located on the inner surface of the section of hollow polymer tubing.

15. A method for removing at least one selected component from a fluid sample to produce a filtered fluid product, comprising the steps of:
a) providing a filter, the filter comprising an inlet, an outlet, and a filter assembly, wherein the filter assembly further comprises an outer housing an a plurality of filter surfaces located within the outer housing;
b) coating the plurality of filter surfaces with an interfacial biomaterial, the interfacial biomaterial comprising a first binding module, configured to selectively bind to the plurality of filter surfaces, and a second binding module, configured to selectively bind to the first binding module and to the at least one selected component;
c) filtering a fluid sample through the filter assembly such that the blood sample comes into contact with the plurality of filter surfaces and such that the at least one selected component is bound to the second binding molecule of the interfacial biomaterial; and
d) collecting the filtered product at the outlet of the filter.

16. The method of claim 15, wherein the providing step further comprises, providing, in the coating, a central macromolecule configured to bind the first binding module to the second binding module.

17. The method of claim 15, further comprising:
a) after the collecting step, flushing the filter with a first eluting compound adapted to elute the at least one selected blood component from the second binding module such that an eluate containing the at least one selected blood component is produced; and
b) collecting the eluate.

18. The method of claim 15, further comprising:
a) after the collecting step, flushing the filter with a first eluting compound adapted to release the first binding module from the second binding module such that an eluate comprising the at least one selected blood component is produced; and
b) collecting the eluate.

19. A method for removing at least two selected components from a fluid sample to produce a filtered fluid product, comprising the steps of:
a) providing a filter, the filter comprising an inlet, an outlet, and a filter assembly, wherein the filter assembly further comprises an outer housing and a plurality of filter surfaces located within the outer housing;
b) coating a first portion of the plurality of filter surfaces with a first interfacial biomaterial, the first interfacial biomaterial comprising a first binding module, configured to selectively bind to the first portion of the plurality of filter surfaces, and a second binding module, configured to selectively bind to the first binding module and to a first selected fluid component;
c) coating a second portion of the plurality of filter surfaces with a second interfacial biomaterial, the first interfacial biomaterial comprising a first binding module, configured to selectively bind to the second portion of the plurality of filter surfaces, and a third binding module, configured to selectively bind to the first binding module and to a second selected fluid component;
d) filtering a fluid sample through the filter assembly such that the fluid sample comes into contact with the first and second portion of the plurality of filter surfaces and such that first selected fluid component is bound to the second binding module of the first interfacial biomaterial and the second selected fluid component is bound to the third binding module of the second interfacial biomaterial; and
e) collecting the filtered fluid product at the outlet of the filter.


SEQUENCE LISTING (462 sequences)

SEQ ID NO: 1 - protein, 18 aa, Artificial Sequence
  Synthetic construct, binding module peptide
CAEKWWWWIQYAWGGVLC

SEQ ID NO: 2 - protein, 18 aa, Artificial Sequence
  Synthetic construct, binding module peptide
CDDIDYIKEAPIDAMMCC

SEQ ID NO: 3 - protein, 18 aa, Artificial Sequence
  Synthetic construct, binding module peptide
CDFFNRHGYNSGCEHSVC

SEQ ID NO: 4 - protein, 18 aa, Artificial Sequence
  Synthetic construct, binding module peptide
CDFHSNKYYINQIAGSDC

SEQ ID NO: 5 - protein, 18 aa, Artificial Sequence
  Synthetic construct, binding module peptide
CDNGLDDCFEPCYWIQLC

SEQ ID NO: 6 - protein, 18 aa, Artificial Sequence
  Synthetic construct, binding module peptide
CFEISSSSTPIELWESVC

SEQ ID NO: 7 - protein, 18 aa, Artificial Sequence
  Synthetic construct, binding module peptide
CFESDFPNVRHHVLKQSC

SEQ ID NO: 8 - protein, 18 aa, Artificial Sequence
  Synthetic construct, binding module peptide
CFFFRRQIEIYYARFGFC

SEQ ID NO: 9 - protein, 18 aa, Artificial Sequence
  Synthetic construct, binding module peptide
CFLFFSMCNMACTKAKEC

SEQ ID NO: 10 - protein, 18 aa, Artificial Sequence
  Synthetic construct, binding module peptide
CFYQNVISSSFAGNPWEC

SEQ ID NO: 11 - protein, 18 aa, Artificial Sequence
  Synthetic construct, binding module peptide
CGDHMTDKNMPNSGISGC

SEQ ID NO: 12 - protein, 18 aa, Artificial Sequence
  Synthetic construct, binding module peptide
CHRYDRRWTMYTRARLRC

SEQ ID NO: 13 - protein, 18 aa, Artificial Sequence
  Synthetic construct, binding module peptide
CIMTSDMVNAAIWNEVQC

SEQ ID NO: 14 - protein, 18 aa, Artificial Sequence
  Synthetic construct, binding module peptide
CLFFFSMIMNFDFPNFEC

SEQ ID NO: 15 - protein, 18 aa, Artificial Sequence
  Synthetic construct, binding module peptide
CLPPPYEPKQLAEPCDGC

SEQ ID NO: 16 - protein, 18 aa, Artificial Sequence
  Synthetic construct, binding module peptide
CLPWYYYYKAQQLYDHYC

SEQ ID NO: 17 - protein, 18 aa, Artificial Sequence
  Synthetic construct, binding module peptide
CMRRWDRWVRWAWSRQKC

SEQ ID NO: 18 - protein, 18 aa, Artificial Sequence
  Synthetic construct, binding module peptide
CMWWWQWGSYIYGELWIC

SEQ ID NO: 19 - protein, 18 aa, Artificial Sequence
  Synthetic construct, binding module peptide
CNEDVNNFPPRMNTELGC

SEQ ID NO: 20 - protein, 18 aa, Artificial Sequence
  Synthetic construct, binding module peptide
CNMLLNSLPLPSEDWSAC

SEQ ID NO: 21 - protein, 18 aa, Artificial Sequence
  Synthetic construct, binding module peptide
CNNNHRDVNWNLRDNTAC

SEQ ID NO: 22 - protein, 18 aa, Artificial Sequence
  Synthetic construct, binding module peptide
CNNNVNWYHYMFIPWAKC

SEQ ID NO: 23 - protein, 18 aa, Artificial Sequence
  Synthetic construct, binding module peptide
CNNVNACQNHENNMHNDC

SEQ ID NO: 24 - protein, 18 aa, Artificial Sequence
  Synthetic construct, binding module peptide
CNPGYNNMMNDSMVMWRC

SEQ ID NO: 25 - protein, 18 aa, Artificial Sequence
  Synthetic construct, binding module peptide
CPFTHSLALNTDRASPGC

SEQ ID NO: 26 - protein, 18 aa, Artificial Sequence
  Synthetic construct, binding module peptide
CPHWPPPWCEWYPENWCC

SEQ ID NO: 27 - protein, 18 aa, Artificial Sequence
  Synthetic construct, binding module peptide
CPNPFPEPLNHDAIDWCC

SEQ ID NO: 28 - protein, 18 aa, Artificial Sequence
  Synthetic construct, binding module peptide
CPNVPRPAQLSICGNLPC

SEQ ID NO: 29 - protein, 18 aa, Artificial Sequence
  Synthetic construct, binding module peptide
CPPMYPQWEGDPNQRYDC

SEQ ID NO: 30 - protein, 18 aa, Artificial Sequence
  Synthetic construct, binding module peptide
CPPPGQVPPWPPSPPPPC

SEQ ID NO: 31 - protein, 18 aa, Artificial Sequence
  Synthetic construct, binding module peptide
CPRRHKRYNWFAHNARMC

SEQ ID NO: 32 - protein, 18 aa, Artificial Sequence
  Synthetic construct, binding module peptide
CRQYRFRPIVRARRLNKC

SEQ ID NO: 33 - protein, 18 aa, Artificial Sequence
  Synthetic construct, binding module peptide
CRRFRSRCPGEWRSWTTC

SEQ ID NO: 34 - protein, 18 aa, Artificial Sequence
  Synthetic construct, binding module peptide
CRVGVRRKEGGFRPWYKC

SEQ ID NO: 35 - protein, 18 aa, Artificial Sequence
  Synthetic construct, binding module peptide
CRVRREPRMRKIKKMALC

SEQ ID NO: 36 - protein, 18 aa, Artificial Sequence
  Synthetic construct, binding module peptide
CRYSTSSWSDMTCGCGQC

SEQ ID NO: 37 - protein, 18 aa, Artificial Sequence
  Synthetic construct, binding module peptide
CSGWKWWVFHVCWKQVHC

SEQ ID NO: 38 - protein, 18 aa, Artificial Sequence
  Synthetic construct, binding module peptide
CSNSSCTSHTLYSSVMGC

SEQ ID NO: 39 - protein, 18 aa, Artificial Sequence
  Synthetic construct, binding module peptide
CSSFMSMHHWHVVVDSCC

SEQ ID NO: 40 - protein, 18 aa, Artificial Sequence
  Synthetic construct, binding module peptide
CSSINSSYVHCLGCTESC

SEQ ID NO: 41 - protein, 18 aa, Artificial Sequence
  Synthetic construct, binding module peptide
CSSRYSTAYHMASNSIFC

SEQ ID NO: 42 - protein, 18 aa, Artificial Sequence
  Synthetic construct, binding module peptide
CTERRRRFNRNRPAKMRC

SEQ ID NO: 43 - protein, 18 aa, Artificial Sequence
  Synthetic construct, binding module peptide
CTPRPPVPVYIPYSSSPC

SEQ ID NO: 44 - protein, 18 aa, Artificial Sequence
  Synthetic construct, binding module peptide
CVDFKSKEKTEIMLRHAC

SEQ ID NO: 45 - protein, 18 aa, Artificial Sequence
  Synthetic construct, binding module peptide
CVFDSKHFSPTHSPHDVC

SEQ ID NO: 46 - protein, 18 aa, Artificial Sequence
  Synthetic construct, binding module peptide
CVYKIYYLYCHPYLTFPC

SEQ ID NO: 47 - protein, 18 aa, Artificial Sequence
  Synthetic construct, binding module peptide
CWKSSSSMMTIVWWNKMC

SEQ ID NO: 48 - protein, 18 aa, Artificial Sequence
  Synthetic construct, binding module peptide
CWMWWPEWWWQCAVQCNC

SEQ ID NO: 49 - protein, 18 aa, Artificial Sequence
  Synthetic construct, binding module peptide
CWYTWWCQASTMGQIYEC

SEQ ID NO: 50 - protein, 18 aa, Artificial Sequence
  Synthetic construct, binding module peptide
CYYDSYPSVPYYYQNPSC

SEQ ID NO: 51 - protein, 18 aa, Artificial Sequence
  Synthetic construct, binding module peptide
CYYFYQALQGLIKNHWAC

SEQ ID NO: 52 - protein, 18 aa, Artificial Sequence
  Synthetic construct, binding module peptide
CYYKPYYPCSAYMNFPLC

SEQ ID NO: 53 - protein, 18 aa, Artificial Sequence
  Synthetic construct, binding module peptide
CYYNGLVVHHSNSGHKDC

SEQ ID NO: 54 - protein, 17 aa, Artificial Sequence
  Synthetic construct, binding module peptide
CANFLSFVNNSYCIDSN

SEQ ID NO: 55 - protein, 17 aa, Artificial Sequence
  Synthetic construct, binding module peptide
CARRRHHHHPPMPHFRR

SEQ ID NO: 56 - protein, 17 aa, Artificial Sequence
  Synthetic construct, binding module peptide
CCDGLITSSWLNWFARG

SEQ ID NO: 57 - protein, 17 aa, Artificial Sequence
  Synthetic construct, binding module peptide
CCEWWWCWKWWQCLWWC

SEQ ID NO: 58 - protein, 17 aa, Artificial Sequence
  Synthetic construct, binding module peptide
CCFNFFTSFNQGKDNFV

SEQ ID NO: 59 - protein, 17 aa, Artificial Sequence
  Synthetic construct, binding module peptide
CCSSCESHWKKFEHNRQ

SEQ ID NO: 60 - protein, 17 aa, Artificial Sequence
  Synthetic construct, binding module peptide
CDDFVLDYDDEYMVMNH

SEQ ID NO: 61 - protein, 17 aa, Artificial Sequence
  Synthetic construct, binding module peptide
CDDMGDDVKDPEDYIDQ

SEQ ID NO: 62 - protein, 17 aa, Artificial Sequence
  Synthetic construct, binding module peptide
CDFCFTNVLFDAFGSHV

SEQ ID NO: 63 - protein, 17 aa, Artificial Sequence
  Synthetic construct, binding module peptide
CDYFSFLECFSNGWSGA

SEQ ID NO: 64 - protein, 17 aa, Artificial Sequence
  Synthetic construct, binding module peptide
CFFFGQGDFMCWICLTV

SEQ ID NO: 65 - protein, 17 aa, Artificial Sequence
  Synthetic construct, binding module peptide
CFFNSFNCTPNEMWYWF

SEQ ID NO: 66 - protein, 17 aa, Artificial Sequence
  Synthetic construct, binding module peptide
CFFSYCFSHDVSTYNTA

SEQ ID NO: 67 - protein, 17 aa, Artificial Sequence
  Synthetic construct, binding module peptide
CFFSYWNCLTNNAFVKP

SEQ ID NO: 68 - protein, 17 aa, Artificial Sequence
  Synthetic construct, binding module peptide
CFGFSDCLSWFVQPSTA

SEQ ID NO: 69 - protein, 17 aa, Artificial Sequence
  Synthetic construct, binding module peptide
CFGNFLSFGFNCESALG

SEQ ID NO: 70 - protein, 17 aa, Artificial Sequence
  Synthetic construct, binding module peptide
CFGNLGNLIYTCDRLMP

SEQ ID NO: 71 - protein, 17 aa, Artificial Sequence
  Synthetic construct, binding module peptide
CFGNVFCVYNQFAAGLF

SEQ ID NO: 72 - protein, 17 aa, Artificial Sequence
  Synthetic construct, binding module peptide
CFTCFSFAFNFCFMCWM

SEQ ID NO: 73 - protein, 17 aa, Artificial Sequence
  Synthetic construct, binding module peptide
CFTFFKASWSWWHHAMM

SEQ ID NO: 74 - protein, 17 aa, Artificial Sequence
  Synthetic construct, binding module peptide
CFVHNFFWFLGKNSNCR

SEQ ID NO: 75 - protein, 17 aa, Artificial Sequence
  Synthetic construct, binding module peptide
CFWYSWLCSASSSDALI

SEQ ID NO: 76 - protein, 17 aa, Artificial Sequence
  Synthetic construct, binding module peptide
CGYFCSFYNYLDIGTAS

SEQ ID NO: 77 - protein, 17 aa, Artificial Sequence
  Synthetic construct, binding module peptide
CHRCKRRHLLRRKQANR

SEQ ID NO: 78 - protein, 17 aa, Artificial Sequence
  Synthetic construct, binding module peptide
CIFNSYFCSFQLTSYGS

SEQ ID NO: 79 - protein, 17 aa, Artificial Sequence
  Synthetic construct, binding module peptide
CKAFFFNFQCFVFVFHF

SEQ ID NO: 80 - protein, 17 aa, Artificial Sequence
  Synthetic construct, binding module peptide
CKFSFDFFARFNRHFYH

SEQ ID NO: 81 - protein, 17 aa, Artificial Sequence
  Synthetic construct, binding module peptide
CKSKKSSHSESEHKKSS

SEQ ID NO: 82 - protein, 17 aa, Artificial Sequence
  Synthetic construct, binding module peptide
CLFNCSGESWPMSIVPS

SEQ ID NO: 83 - protein, 17 aa, Artificial Sequence
  Synthetic construct, binding module peptide
CLKDYYYSPCSYSCDQH

SEQ ID NO: 84 - protein, 17 aa, Artificial Sequence
  Synthetic construct, binding module peptide
CLLKYCYSDLASSSLSI

SEQ ID NO: 85 - protein, 17 aa, Artificial Sequence
  Synthetic construct, binding module peptide
CLVFMRPYFLLVFLMCW

SEQ ID NO: 86 - protein, 17 aa, Artificial Sequence
  Synthetic construct, binding module peptide
CLYCHLNNQFLSWVSGN

SEQ ID NO: 87 - protein, 17 aa, Artificial Sequence
  Synthetic construct, binding module peptide
CLYCLNYANFSDPMTMF

SEQ ID NO: 88 - protein, 17 aa, Artificial Sequence
  Synthetic construct, binding module peptide
CNHLGFFSSFCDRLVEN

SEQ ID NO: 89 - protein, 17 aa, Artificial Sequence
  Synthetic construct, binding module peptide
CNSFMFINGSFKETGGC

SEQ ID NO: 90 - protein, 17 aa, Artificial Sequence
  Synthetic construct, binding module peptide
CNSSSYSWYCWFGGSSP

SEQ ID NO: 91 - protein, 17 aa, Artificial Sequence
  Synthetic construct, binding module peptide
CRDRQRWVRIFNRRCVT

SEQ ID NO: 92 - protein, 17 aa, Artificial Sequence
  Synthetic construct, binding module peptide
CRMKKRRRAHPPRNCME

SEQ ID NO: 93 - protein, 17 aa, Artificial Sequence
  Synthetic construct, binding module peptide
CRRMRCRDHTQKWRRER

SEQ ID NO: 94 - protein, 17 aa, Artificial Sequence
  Synthetic construct, binding module peptide
CRRRKNFQRCFRPLLYP

SEQ ID NO: 95 - protein, 17 aa, Artificial Sequence
  Synthetic construct, binding module peptide
CRRRSQRRNRRGNDDSA

SEQ ID NO: 96 - protein, 17 aa, Artificial Sequence
  Synthetic construct, binding module peptide
CSFFMPWCNFLNGEMAV

SEQ ID NO: 97 - protein, 17 aa, Artificial Sequence
  Synthetic construct, binding module peptide
CSFSVSKSSQIFAVSYS

SEQ ID NO: 98 - protein, 17 aa, Artificial Sequence
  Synthetic construct, binding module peptide
CSLTGCLYDYVSFGWGA

SEQ ID NO: 99 - protein, 17 aa, Artificial Sequence
  Synthetic construct, binding module peptide
CSSSMTYRTSSSWHLKI

SEQ ID NO: 100 - protein, 17 aa, Artificial Sequence
  Synthetic construct, binding module peptide
CSTSYSWNKWQISISSY

SEQ ID NO: 101 - protein, 17 aa, Artificial Sequence
  Synthetic construct, binding module peptide
CTCFNLFDMKTCPSFCT

SEQ ID NO: 102 - protein, 17 aa, Artificial Sequence
  Synthetic construct, binding module peptide
CTFGFPCVMSLVNHVPS

SEQ ID NO: 103 - protein, 17 aa, Artificial Sequence
  Synthetic construct, binding module peptide
CTNSNLNSSSWHTMVDR

SEQ ID NO: 104 - protein, 17 aa, Artificial Sequence
  Synthetic construct, binding module peptide
CTWWWWWVVNREPYVAC

SEQ ID NO: 105 - protein, 17 aa, Artificial Sequence
  Synthetic construct, binding module peptide
CWDWMTWGNDVLVNTDW

SEQ ID NO: 106 - protein, 17 aa, Artificial Sequence
  Synthetic construct, binding module peptide
CWLDDDSDDYDDDDMMA

SEQ ID NO: 107 - protein, 17 aa, Artificial Sequence
  Synthetic construct, binding module peptide
CWMGLFECPDAWLHDWD

SEQ ID NO: 108 - protein, 17 aa, Artificial Sequence
  Synthetic construct, binding module peptide
CWNISCMFGFGWGGGGL

SEQ ID NO: 109 - protein, 17 aa, Artificial Sequence
  Synthetic construct, binding module peptide
CYAYYFFFYSSGRGYHQ

SEQ ID NO: 110 - protein, 17 aa, Artificial Sequence
  Synthetic construct, binding module peptide
CYFPFYCYNTSSLSLDF

SEQ ID NO: 111 - protein, 16 aa, Artificial Sequence
  Synthetic construct, binding module peptide
ADRVWPRHTSSPYHRH

SEQ ID NO: 112 - protein, 16 aa, Artificial Sequence
  Synthetic construct, binding module peptide
AFISNLHAACSVGSCK

SEQ ID NO: 113 - protein, 16 aa, Artificial Sequence
  Synthetic construct, binding module peptide
CHTPWPPMNRYASVLI

SEQ ID NO: 114 - protein, 16 aa, Artificial Sequence
  Synthetic construct, binding module peptide
CTRRRRFCVIIFRREM

SEQ ID NO: 115 - protein, 16 aa, Artificial Sequence
  Synthetic construct, binding module peptide
CTSSSQKHCYHGHSSD

SEQ ID NO: 116 - protein, 16 aa, Artificial Sequence
  Synthetic construct, binding module peptide
DCCCMWDDGVGDDVDM

SEQ ID NO: 117 - protein, 16 aa, Artificial Sequence
  Synthetic construct, binding module peptide
DFCFMMMNCTMNAHYF

SEQ ID NO: 118 - protein, 16 aa, Artificial Sequence
  Synthetic construct, binding module peptide
DVNSIWMSRVIEWTYD

SEQ ID NO: 119 - protein, 16 aa, Artificial Sequence
  Synthetic construct, binding module peptide
DWCNNAWDTYAIHNDC

SEQ ID NO: 120 - protein, 16 aa, Artificial Sequence
  Synthetic construct, binding module peptide
FLFFTNMVWYFFIMGA

SEQ ID NO: 121 - protein, 16 aa, Artificial Sequence
  Synthetic construct, binding module peptide
FTVSSHIIEWSADSVV

SEQ ID NO: 122 - protein, 16 aa, Artificial Sequence
  Synthetic construct, binding module peptide
GAGGFFLPCLWNPDRT

SEQ ID NO: 123 - protein, 16 aa, Artificial Sequence
  Synthetic construct, binding module peptide
GKCVFRREDCFWYYMH

SEQ ID NO: 124 - protein, 16 aa, Artificial Sequence
  Synthetic construct, binding module peptide
GSSSCQGVSGSDYVMK

SEQ ID NO: 125 - protein, 16 aa, Artificial Sequence
  Synthetic construct, binding module peptide
HASIHHCSYQGYGQSG

SEQ ID NO: 126 - protein, 16 aa, Artificial Sequence
  Synthetic construct, binding module peptide
HCNNENRWHHNGAIGV

SEQ ID NO: 127 - protein, 16 aa, Artificial Sequence
  Synthetic construct, binding module peptide
HISSCQMVQSWSRPAH

SEQ ID NO: 128 - protein, 16 aa, Artificial Sequence
  Synthetic construct, binding module peptide
IWEWFELEMLYVNRYC

SEQ ID NO: 129 - protein, 16 aa, Artificial Sequence
  Synthetic construct, binding module peptide
LIHRYCRRVPCRRELK

SEQ ID NO: 130 - protein, 16 aa, Artificial Sequence
  Synthetic construct, binding module peptide
MSNFLIEFTYDNVGVR

SEQ ID NO: 131 - protein, 16 aa, Artificial Sequence
  Synthetic construct, binding module peptide
NFFVEWAFDTQDREEL

SEQ ID NO: 132 - protein, 16 aa, Artificial Sequence
  Synthetic construct, binding module peptide
NGNENDTINDNDINAS

SEQ ID NO: 133 - protein, 16 aa, Artificial Sequence
  Synthetic construct, binding module peptide
NINIVEERFMVEWDVQ

SEQ ID NO: 134 - protein, 16 aa, Artificial Sequence
  Synthetic construct, binding module peptide
NPWASSLVAACYLDES

SEQ ID NO: 135 - protein, 16 aa, Artificial Sequence
  Synthetic construct, binding module peptide
NWWMVNLIPDEWCWNS

SEQ ID NO: 136 - protein, 16 aa, Artificial Sequence
  Synthetic construct, binding module peptide
PFLFEASDRHPAFNHM

SEQ ID NO: 137 - protein, 16 aa, Artificial Sequence
  Synthetic construct, binding module peptide
PGSSTFYSITMTWDLP

SEQ ID NO: 138 - protein, 16 aa, Artificial Sequence
  Synthetic construct, binding module peptide
PPSSNSNFMLEFSWDS

SEQ ID NO: 139 - protein, 16 aa, Artificial Sequence
  Synthetic construct, binding module peptide
PQSEHSKSYMSWARSS

SEQ ID NO: 140 - protein, 16 aa, Artificial Sequence
  Synthetic construct, binding module peptide
PSACSRRIIQDTFFFM

SEQ ID NO: 141 - protein, 16 aa, Artificial Sequence
  Synthetic construct, binding module peptide
QELRVRKRRRPKDHER

SEQ ID NO: 142 - protein, 16 aa, Artificial Sequence
  Synthetic construct, binding module peptide
QEMLNFFFHNGNFFFV

SEQ ID NO: 143 - protein, 16 aa, Artificial Sequence
  Synthetic construct, binding module peptide
QHRQHHNVIYSAVCVA

SEQ ID NO: 144 - protein, 16 aa, Artificial Sequence
  Synthetic construct, binding module peptide
QMDTIDDMTWTGDDDC

SEQ ID NO: 145 - protein, 16 aa, Artificial Sequence
  Synthetic construct, binding module peptide
RGPYIWWLEEQSRTWE

SEQ ID NO: 146 - protein, 16 aa, Artificial Sequence
  Synthetic construct, binding module peptide
RRRNKLARTLVYRRRV

SEQ ID NO: 147 - protein, 16 aa, Artificial Sequence
  Synthetic construct, binding module peptide
RRRPKPGPHIIFTAIN

SEQ ID NO: 148 - protein, 16 aa, Artificial Sequence
  Synthetic construct, binding module peptide
RRYATWSVASIQECPR

SEQ ID NO: 149 - protein, 16 aa, Artificial Sequence
  Synthetic construct, binding module peptide
RYPYDMDWDWHHQERD

SEQ ID NO: 150 - protein, 16 aa, Artificial Sequence
  Synthetic construct, binding module peptide
SFFFWDTFGESNKFFM

SEQ ID NO: 151 - protein, 16 aa, Artificial Sequence
  Synthetic construct, binding module peptide
SFMFNDSIDDDDDVSE

SEQ ID NO: 152 - protein, 16 aa, Artificial Sequence
  Synthetic construct, binding module peptide
SPQARSHEDQVMQWWI

SEQ ID NO: 153 - protein, 16 aa, Artificial Sequence
  Synthetic construct, binding module peptide
TFDDAMLEWSLVEWDI

SEQ ID NO: 154 - protein, 16 aa, Artificial Sequence
  Synthetic construct, binding module peptide
TGQSSMVNHMVSENGG

SEQ ID NO: 155 - protein, 16 aa, Artificial Sequence
  Synthetic construct, binding module peptide
TMQDFSSDEFYTWTWD

SEQ ID NO: 156 - protein, 16 aa, Artificial Sequence
  Synthetic construct, binding module peptide
VFGFSCFEKDKRFDEL

SEQ ID NO: 157 - protein, 16 aa, Artificial Sequence
  Synthetic construct, binding module peptide
VLGWKSWKIYWAWLVE

SEQ ID NO: 158 - protein, 16 aa, Artificial Sequence
  Synthetic construct, binding module peptide
WLWTWQETAEHPIWNS

SEQ ID NO: 159 - protein, 16 aa, Artificial Sequence
  Synthetic construct, binding module peptide
WMWQICPCMMHWVLNW

SEQ ID NO: 160 - protein, 16 aa, Artificial Sequence
  Synthetic construct, binding module peptide
WNCDYETGAGWRCSEA

SEQ ID NO: 161 - protein, 16 aa, Artificial Sequence
  Synthetic construct, binding module peptide
WNFYFVAFIALPMEFV

SEQ ID NO: 162 - protein, 16 aa, Artificial Sequence
  Synthetic construct, binding module peptide
WWFRFKRRRRWMKSVR

SEQ ID NO: 163 - protein, 16 aa, Artificial Sequence
  Synthetic construct, binding module peptide
YDMMMDMLKNDDKGFF

SEQ ID NO: 164 - protein, 16 aa, Artificial Sequence
  Synthetic construct, binding module peptide
YRMADRDVHRWDKEYE

SEQ ID NO: 165 - protein, 16 aa, Artificial Sequence
  Synthetic construct, binding module peptide
YRNMERSNMAETNILA

SEQ ID NO: 166 - protein, 16 aa, Artificial Sequence
  Synthetic construct, binding module peptide
YYFTEWSEDTSGGSSG

SEQ ID NO: 167 - protein, 13 aa, Artificial Sequence
  Synthetic construct, binding module peptide
AKILYYYDMQWHI

SEQ ID NO: 168 - protein, 13 aa, Artificial Sequence
  Synthetic construct, binding module peptide
APFLVWYASTSDT

SEQ ID NO: 169 - protein, 13 aa, Artificial Sequence
  Synthetic construct, binding module peptide
AVSTALYNTWQVL

SEQ ID NO: 170 - protein, 13 aa, Artificial Sequence
  Synthetic construct, binding module peptide
CAHPPPYKENYLY

SEQ ID NO: 171 - protein, 13 aa, Artificial Sequence
  Synthetic construct, binding module peptide
CCWTEAYDAHPWR

SEQ ID NO: 172 - protein, 13 aa, Artificial Sequence
  Synthetic construct, binding module peptide
CKFFFHYHIGFAT

SEQ ID NO: 173 - protein, 13 aa, Artificial Sequence
  Synthetic construct, binding module peptide
CVWCSEYFREDPP

SEQ ID NO: 174 - protein, 13 aa, Artificial Sequence
  Synthetic construct, binding module peptide
CYTSKYYREKYEL

SEQ ID NO: 175 - protein, 13 aa, Artificial Sequence
  Synthetic construct, binding module peptide
DTIWWWYMWCWHY

SEQ ID NO: 176 - protein, 13 aa, Artificial Sequence
  Synthetic construct, binding module peptide
EHGPFVDSEYPQP

SEQ ID NO: 177 - protein, 13 aa, Artificial Sequence
  Synthetic construct, binding module peptide
FADNLGYVGSDVI

SEQ ID NO: 178 - protein, 13 aa, Artificial Sequence
  Synthetic construct, binding module peptide
FAPMKSYGVSLPP

SEQ ID NO: 179 - protein, 13 aa, Artificial Sequence
  Synthetic construct, binding module peptide
FELATGYVPALLK

SEQ ID NO: 180 - protein, 13 aa, Artificial Sequence
  Synthetic construct, binding module peptide
FFFSMSYFFFRAA

SEQ ID NO: 181 - protein, 13 aa, Artificial Sequence
  Synthetic construct, binding module peptide
FFGFDVYDMSNAL

SEQ ID NO: 182 - protein, 13 aa, Artificial Sequence
  Synthetic construct, binding module peptide
FFHFCFYTCMFHL

SEQ ID NO: 183 - protein, 13 aa, Artificial Sequence
  Synthetic construct, binding module peptide
FFLSPFYFFNEFF

SEQ ID NO: 184 - protein, 13 aa, Artificial Sequence
  Synthetic construct, binding module peptide
FFMASSYSYPVAG

SEQ ID NO: 185 - protein, 13 aa, Artificial Sequence
  Synthetic construct, binding module peptide
FFPSSWYSHLGVL

SEQ ID NO: 186 - protein, 13 aa, Artificial Sequence
  Synthetic construct, binding module peptide
FFVLFLYLWLGVS

SEQ ID NO: 187 - protein, 13 aa, Artificial Sequence
  Synthetic construct, binding module peptide
FGCELPYSGVCSV

SEQ ID NO: 188 - protein, 13 aa, Artificial Sequence
  Synthetic construct, binding module peptide
FGSDVFYLRSAPH

SEQ ID NO: 189 - protein, 13 aa, Artificial Sequence
  Synthetic construct, binding module peptide
FHEAPVYETSEPP

SEQ ID NO: 190 - protein, 13 aa, Artificial Sequence
  Synthetic construct, binding module peptide
FLGFQDYKSAAMM

SEQ ID NO: 191 - protein, 13 aa, Artificial Sequence
  Synthetic construct, binding module peptide
FLLTGEYVDVVAA

SEQ ID NO: 192 - protein, 13 aa, Artificial Sequence
  Synthetic construct, binding module peptide
FLSFANYEDELLR

SEQ ID NO: 193 - protein, 13 aa, Artificial Sequence
  Synthetic construct, binding module peptide
FMFIFFYPVFCFQ

SEQ ID NO: 194 - protein, 13 aa, Artificial Sequence
  Synthetic construct, binding module peptide
FRFFNHYRYPSGQ

SEQ ID NO: 195 - protein, 13 aa, Artificial Sequence
  Synthetic construct, binding module peptide
FRMDFDYLYPSLP

SEQ ID NO: 196 - protein, 13 aa, Artificial Sequence
  Synthetic construct, binding module peptide
FRYFYFYSHGFKF

SEQ ID NO: 197 - protein, 13 aa, Artificial Sequence
  Synthetic construct, binding module peptide
FSALPTYEVNSYK

SEQ ID NO: 198 - protein, 13 aa, Artificial Sequence
  Synthetic construct, binding module peptide
FSDSSFYSDLSVV

SEQ ID NO: 199 - protein, 13 aa, Artificial Sequence
  Synthetic construct, binding module peptide
FSSVDSYSGPRPD

SEQ ID NO: 200 - protein, 13 aa, Artificial Sequence
  Synthetic construct, binding module peptide
FSYSVSYAHPEGL

SEQ ID NO: 201 - protein, 13 aa, Artificial Sequence
  Synthetic construct, binding module peptide
FVGFFLYLTLLLP

SEQ ID NO: 202 - protein, 13 aa, Artificial Sequence
  Synthetic construct, binding module peptide
GENFCPYSFFGCG

SEQ ID NO: 203 - protein, 13 aa, Artificial Sequence
  Synthetic construct, binding module peptide
GFAWSSYLGTTVH

SEQ ID NO: 204 - protein, 13 aa, Artificial Sequence
  Synthetic construct, binding module peptide
GFPFIFYVVDWMR

SEQ ID NO: 205 - protein, 13 aa, Artificial Sequence
  Synthetic construct, binding module peptide
GFSEFLYDLEVGI

SEQ ID NO: 206 - protein, 13 aa, Artificial Sequence
  Synthetic construct, binding module peptide
GFVAYNYDKYSGA

SEQ ID NO: 207 - protein, 13 aa, Artificial Sequence
  Synthetic construct, binding module peptide
GVSQFLYDWVKGG

SEQ ID NO: 208 - protein, 13 aa, Artificial Sequence
  Synthetic construct, binding module peptide
GYNIYWYINNVEY

SEQ ID NO: 209 - protein, 13 aa, Artificial Sequence
  Synthetic construct, binding module peptide
HYKYNVYCKYNGY

SEQ ID NO: 210 - protein, 13 aa, Artificial Sequence
  Synthetic construct, binding module peptide
IFLPWHYDGYTFA

SEQ ID NO: 211 - protein, 13 aa, Artificial Sequence
  Synthetic construct, binding module peptide
IFSFLSYVPVDKV

SEQ ID NO: 212 - protein, 13 aa, Artificial Sequence
  Synthetic construct, binding module peptide
IYAALYYRFPTMD

SEQ ID NO: 213 - protein, 13 aa, Artificial Sequence
  Synthetic construct, binding module peptide
KFFFWFYINFVMM

SEQ ID NO: 214 - protein, 13 aa, Artificial Sequence
  Synthetic construct, binding module peptide
LDPLVPYLYENLF

SEQ ID NO: 215 - protein, 13 aa, Artificial Sequence
  Synthetic construct, binding module peptide
LFDAYWYSDTAMS

SEQ ID NO: 216 - protein, 13 aa, Artificial Sequence
  Synthetic construct, binding module peptide
LLFFDDYFKSAGR

SEQ ID NO: 217 - protein, 13 aa, Artificial Sequence
  Synthetic construct, binding module peptide
LNFMIFYLSLNPW

SEQ ID NO: 218 - protein, 13 aa, Artificial Sequence
  Synthetic construct, binding module peptide
LPHLIQYRVLLVS

SEQ ID NO: 219 - protein, 13 aa, Artificial Sequence
  Synthetic construct, binding module peptide
LPSQFGYGSVPTD

SEQ ID NO: 220 - protein, 13 aa, Artificial Sequence
  Synthetic construct, binding module peptide
LPSQFGYGSVPTD

SEQ ID NO: 221 - protein, 13 aa, Artificial Sequence
  Synthetic construct, binding module peptide
LSFSDFYFSEGSE

SEQ ID NO: 222 - protein, 13 aa, Artificial Sequence
  Synthetic construct, binding module peptide
LTNSGVYDGTPLP

SEQ ID NO: 223 - protein, 13 aa, Artificial Sequence
  Synthetic construct, binding module peptide
LVLLILYLFLSWP

SEQ ID NO: 224 - protein, 13 aa, Artificial Sequence
  Synthetic construct, binding module peptide
LVLLLFYFLMLSP

SEQ ID NO: 225 - protein, 13 aa, Artificial Sequence
  Synthetic construct, binding module peptide
LYLFYPYPNYYMV

SEQ ID NO: 226 - protein, 13 aa, Artificial Sequence
  Synthetic construct, binding module peptide
NFSSSFYSLVSEG

SEQ ID NO: 227 - protein, 13 aa, Artificial Sequence
  Synthetic construct, binding module peptide
NWYAEYYYVYDKG

SEQ ID NO: 228 - protein, 13 aa, Artificial Sequence
  Synthetic construct, binding module peptide
NYFSAMYYDGWMS

SEQ ID NO: 229 - protein, 13 aa, Artificial Sequence
  Synthetic construct, binding module peptide
PASLELYENLVAG

SEQ ID NO: 230 - protein, 13 aa, Artificial Sequence
  Synthetic construct, binding module peptide
PCWYRYYHEFWIW

SEQ ID NO: 231 - protein, 13 aa, Artificial Sequence
  Synthetic construct, binding module peptide
PLYYESYRMRTYQ

SEQ ID NO: 232 - protein, 13 aa, Artificial Sequence
  Synthetic construct, binding module peptide
QYASYMYYCFPKY

SEQ ID NO: 233 - protein, 13 aa, Artificial Sequence
  Synthetic construct, binding module peptide
RAWWWWYLDMYWT

SEQ ID NO: 234 - protein, 13 aa, Artificial Sequence
  Synthetic construct, binding module peptide
RAYNYYYYVMYAC

SEQ ID NO: 235 - protein, 13 aa, Artificial Sequence
  Synthetic construct, binding module peptide
RWIWWPYVNMIWT

SEQ ID NO: 236 - protein, 13 aa, Artificial Sequence
  Synthetic construct, binding module peptide
SDFLSPYLAYERS

SEQ ID NO: 237 - protein, 13 aa, Artificial Sequence
  Synthetic construct, binding module peptide
SFDVRSYVLAGTE

SEQ ID NO: 238 - protein, 13 aa, Artificial Sequence
  Synthetic construct, binding module peptide
SLFLDDYALGPRV

SEQ ID NO: 239 - protein, 13 aa, Artificial Sequence
  Synthetic construct, binding module peptide
SSVLGFYDPVEVS

SEQ ID NO: 240 - protein, 13 aa, Artificial Sequence
  Synthetic construct, binding module peptide
SVAFYDYLPTDLP

SEQ ID NO: 241 - protein, 13 aa, Artificial Sequence
  Synthetic construct, binding module peptide
SVLDFNYGHDVNV

SEQ ID NO: 242 - protein, 13 aa, Artificial Sequence
  Synthetic construct, binding module peptide
SVSDFLYRSIYSL

SEQ ID NO: 243 - protein, 13 aa, Artificial Sequence
  Synthetic construct, binding module peptide
SVSDFLYRSIYSL

SEQ ID NO: 244 - protein, 13 aa, Artificial Sequence
  Synthetic construct, binding module peptide
SVSDFLYRSIYSL

SEQ ID NO: 245 - protein, 13 aa, Artificial Sequence
  Synthetic construct, binding module peptide
SWSWWRYGPQNTV

SEQ ID NO: 246 - protein, 13 aa, Artificial Sequence
  Synthetic construct, binding module peptide
SYGFPIYDALLEQ

SEQ ID NO: 247 - protein, 13 aa, Artificial Sequence
  Synthetic construct, binding module peptide
VFDVGLYWHAAPP

SEQ ID NO: 248 - protein, 13 aa, Artificial Sequence
  Synthetic construct, binding module peptide
VGFWVDYDNSSVM

SEQ ID NO: 249 - protein, 13 aa, Artificial Sequence
  Synthetic construct, binding module peptide
VLDLPYYWPVKYT

SEQ ID NO: 250 - protein, 13 aa, Artificial Sequence
  Synthetic construct, binding module peptide
VLLADSYQRDEHM

SEQ ID NO: 251 - protein, 13 aa, Artificial Sequence
  Synthetic construct, binding module peptide
VLLFDDYGYAESA

SEQ ID NO: 252 - protein, 13 aa, Artificial Sequence
  Synthetic construct, binding module peptide
VSASGMYDGVDLM

SEQ ID NO: 253 - protein, 13 aa, Artificial Sequence
  Synthetic construct, binding module peptide
VSLLFSYSPAGYD

SEQ ID NO: 254 - protein, 13 aa, Artificial Sequence
  Synthetic construct, binding module peptide
VSSEWTYGAVADL

SEQ ID NO: 255 - protein, 13 aa, Artificial Sequence
  Synthetic construct, binding module peptide
VSVLSDYSIKALL

SEQ ID NO: 256 - protein, 13 aa, Artificial Sequence
  Synthetic construct, binding module peptide
WADMYYYYDWYTM

SEQ ID NO: 257 - protein, 13 aa, Artificial Sequence
  Synthetic construct, binding module peptide
WDWWQFYEKMWLF

SEQ ID NO: 258 - protein, 13 aa, Artificial Sequence
  Synthetic construct, binding module peptide
WNWWGVYLGICWL

SEQ ID NO: 259 - protein, 13 aa, Artificial Sequence
  Synthetic construct, binding module peptide
WWQTWWYRTYWEI

SEQ ID NO: 260 - protein, 13 aa, Artificial Sequence
  Synthetic construct, binding module peptide
YAGVYSYFTGSTL

SEQ ID NO: 261 - protein, 13 aa, Artificial Sequence
  Synthetic construct, binding module peptide
YCQYREYYTMYVC

SEQ ID NO: 262 - protein, 13 aa, Artificial Sequence
  Synthetic construct, binding module peptide
YFVETYYNRYHVS

SEQ ID NO: 263 - protein, 13 aa, Artificial Sequence
  Synthetic construct, binding module peptide
YLSLHAYESFGGS

SEQ ID NO: 264 - protein, 13 aa, Artificial Sequence
  Synthetic construct, binding module peptide
YRYQMSYYAYQYH

SEQ ID NO: 265 - protein, 13 aa, Artificial Sequence
  Synthetic construct, binding module peptide
YSMYPIYNKCSQH

SEQ ID NO: 266 - protein, 13 aa, Artificial Sequence
  Synthetic construct, binding module peptide
YWIYNNYTYYYCG

SEQ ID NO: 267 - protein, 13 aa, Artificial Sequence
  Synthetic construct, binding module peptide
YWWEQWYSWWIEH

SEQ ID NO: 268 - protein, 13 aa, Artificial Sequence
  Synthetic construct, binding module peptide
YYRDASYTYPYMY

SEQ ID NO: 269 - protein, 13 aa, Artificial Sequence
  Synthetic construct, binding module peptide
YYYIPVYSAQCYT

SEQ ID NO: 270 - protein, 13 aa, Artificial Sequence
  Synthetic construct, binding module peptide
ACPWPIPPWPLRV

SEQ ID NO: 271 - protein, 13 aa, Artificial Sequence
  Synthetic construct, binding module peptide
ARRWPLPRRDQFS

SEQ ID NO: 272 - protein, 13 aa, Artificial Sequence
  Synthetic construct, binding module peptide
CRRIQQPCVFRRH

SEQ ID NO: 273 - protein, 13 aa, Artificial Sequence
  Synthetic construct, binding module peptide
DEPPCAPECNGDG

SEQ ID NO: 274 - protein, 13 aa, Artificial Sequence
  Synthetic construct, binding module peptide
DFQFPKPAFCSTC

SEQ ID NO: 275 - protein, 13 aa, Artificial Sequence
  Synthetic construct, binding module peptide
ELYFFFPCGSFCQ

SEQ ID NO: 276 - protein, 13 aa, Artificial Sequence
  Synthetic construct, binding module peptide
FFGFNHPFLFSCW

SEQ ID NO: 277 - protein, 13 aa, Artificial Sequence
  Synthetic construct, binding module peptide
FFQSIQPIFARSM

SEQ ID NO: 278 - protein, 13 aa, Artificial Sequence
  Synthetic construct, binding module peptide
FFWVKDPSPCFDH

SEQ ID NO: 279 - protein, 13 aa, Artificial Sequence
  Synthetic construct, binding module peptide
FGKFFDPLRRAKD

SEQ ID NO: 280 - protein, 13 aa, Artificial Sequence
  Synthetic construct, binding module peptide
FKGEFWPAFGVQV

SEQ ID NO: 281 - protein, 13 aa, Artificial Sequence
  Synthetic construct, binding module peptide
FKLHWFPTCPFIQ

SEQ ID NO: 282 - protein, 13 aa, Artificial Sequence
  Synthetic construct, binding module peptide
FLSFVFPASAWGG

SEQ ID NO: 283 - protein, 13 aa, Artificial Sequence
  Synthetic construct, binding module peptide
FMDIWSPWHLLGT

SEQ ID NO: 284 - protein, 13 aa, Artificial Sequence
  Synthetic construct, binding module peptide
FNPPEPPCPEFSK

SEQ ID NO: 285 - protein, 13 aa, Artificial Sequence
  Synthetic construct, binding module peptide
FQFFDPPSFFGFK

SEQ ID NO: 286 - protein, 13 aa, Artificial Sequence
  Synthetic construct, binding module peptide
FQFSFQPDGVERR

SEQ ID NO: 287 - protein, 13 aa, Artificial Sequence
  Synthetic construct, binding module peptide
FQNCFWPIFEAME

SEQ ID NO: 288 - protein, 13 aa, Artificial Sequence
  Synthetic construct, binding module peptide
FSFFADPIELEWD

SEQ ID NO: 289 - protein, 12 aa, Artificial Sequence
  Synthetic construct, binding module peptide
FSSLFFPHWAQL

SEQ ID NO: 290 - protein, 13 aa, Artificial Sequence
  Synthetic construct, binding module peptide
FYMPFGPTWWQHV

SEQ ID NO: 291 - protein, 13 aa, Artificial Sequence
  Synthetic construct, binding module peptide
FYYFGFPQCLILF

SEQ ID NO: 292 - protein, 13 aa, Artificial Sequence
  Synthetic construct, binding module peptide
GFEEFQPVDFIIR

SEQ ID NO: 293 - protein, 13 aa, Artificial Sequence
  Synthetic construct, binding module peptide
GLTRFFPVSFSFF

SEQ ID NO: 294 - protein, 13 aa, Artificial Sequence
  Synthetic construct, binding module peptide
HARPPCPFVNEKP

SEQ ID NO: 295 - protein, 13 aa, Artificial Sequence
  Synthetic construct, binding module peptide
HEFMWFPVHWEFH

SEQ ID NO: 296 - protein, 13 aa, Artificial Sequence
  Synthetic construct, binding module peptide
HRNPRRPQIEGVR

SEQ ID NO: 297 - protein, 13 aa, Artificial Sequence
  Synthetic construct, binding module peptide
ISGHCFPCIEVSD

SEQ ID NO: 298 - protein, 13 aa, Artificial Sequence
  Synthetic construct, binding module peptide
KFQDFMPQMFHGI

SEQ ID NO: 299 - protein, 13 aa, Artificial Sequence
  Synthetic construct, binding module peptide
LFFMPFPFFFFPY

SEQ ID NO: 300 - protein, 13 aa, Artificial Sequence
  Synthetic construct, binding module peptide
LFSWFLPTDNYPV

SEQ ID NO: 301 - protein, 13 aa, Artificial Sequence
  Synthetic construct, binding module peptide
LVCIRRPRRRCFC

SEQ ID NO: 302 - protein, 13 aa, Artificial Sequence
  Synthetic construct, binding module peptide
MPRRERPLWMLTR

SEQ ID NO: 303 - protein, 13 aa, Artificial Sequence
  Synthetic construct, binding module peptide
MRRHRAPRSQCME

SEQ ID NO: 304 - protein, 13 aa, Artificial Sequence
  Synthetic construct, binding module peptide
NFFGPIPMNFAFT

SEQ ID NO: 305 - protein, 13 aa, Artificial Sequence
  Synthetic construct, binding module peptide
NFFSIDPFCQAIY

SEQ ID NO: 306 - protein, 13 aa, Artificial Sequence
  Synthetic construct, binding module peptide
NNGARRPYVASNP

SEQ ID NO: 307 - protein, 13 aa, Artificial Sequence
  Synthetic construct, binding module peptide
NRRRYRPRFYRRC

SEQ ID NO: 308 - protein, 13 aa, Artificial Sequence
  Synthetic construct, binding module peptide
PFFWMFPICFPPN

SEQ ID NO: 309 - protein, 13 aa, Artificial Sequence
  Synthetic construct, binding module peptide
PFGLFPPQVYYFL

SEQ ID NO: 310 - protein, 13 aa, Artificial Sequence
  Synthetic construct, binding module peptide
PGAAPPPCNNSDN

SEQ ID NO: 311 - protein, 13 aa, Artificial Sequence
  Synthetic construct, binding module peptide
PPCPWRPSATHLP

SEQ ID NO: 312 - protein, 13 aa, Artificial Sequence
  Synthetic construct, binding module peptide
PPKFLAPHTSAML

SEQ ID NO: 313 - protein, 13 aa, Artificial Sequence
  Synthetic construct, binding module peptide
PPRVAFPIRQRRV

SEQ ID NO: 314 - protein, 13 aa, Artificial Sequence
  Synthetic construct, binding module peptide
PTRPNGPESEDLF

SEQ ID NO: 315 - protein, 13 aa, Artificial Sequence
  Synthetic construct, binding module peptide
QCPDPSPSKCPFG

SEQ ID NO: 316 - protein, 13 aa, Artificial Sequence
  Synthetic construct, binding module peptide
QRRAPRPSEHRRE

SEQ ID NO: 317 - protein, 13 aa, Artificial Sequence
  Synthetic construct, binding module peptide
RARRAGPLGDRKL

SEQ ID NO: 318 - protein, 13 aa, Artificial Sequence
  Synthetic construct, binding module peptide
REGRTRPRYPRWF

SEQ ID NO: 319 - protein, 13 aa, Artificial Sequence
  Synthetic construct, binding module peptide
REPNPPPLQSPMS

SEQ ID NO: 320 - protein, 13 aa, Artificial Sequence
  Synthetic construct, binding module peptide
RGFQFGPSTFEYF

SEQ ID NO: 321 - protein, 13 aa, Artificial Sequence
  Synthetic construct, binding module peptide
RGPRRTPTIHRPW

SEQ ID NO: 322 - protein, 13 aa, Artificial Sequence
  Synthetic construct, binding module peptide
RHFHVRPVNWWSK

SEQ ID NO: 323 - protein, 13 aa, Artificial Sequence
  Synthetic construct, binding module peptide
RINRSRPIMWQRT

SEQ ID NO: 324 - protein, 13 aa, Artificial Sequence
  Synthetic construct, binding module peptide
RNDRVRPWKVKHQ

SEQ ID NO: 325 - protein, 13 aa, Artificial Sequence
  Synthetic construct, binding module peptide
RNMRYRPQYADLC

SEQ ID NO: 326 - protein, 13 aa, Artificial Sequence
  Synthetic construct, binding module peptide
RNNRPKPTQSHRV

SEQ ID NO: 327 - protein, 13 aa, Artificial Sequence
  Synthetic construct, binding module peptide
RRHRWWPQEFSRH

SEQ ID NO: 328 - protein, 13 aa, Artificial Sequence
  Synthetic construct, binding module peptide
RRRLFTPNSRARH

SEQ ID NO: 329 - protein, 13 aa, Artificial Sequence
  Synthetic construct, binding module peptide
RRSRFVPEYLFRP

SEQ ID NO: 330 - protein, 13 aa, Artificial Sequence
  Synthetic construct, binding module peptide
RWHPRYPVMKKNS

SEQ ID NO: 331 - protein, 13 aa, Artificial Sequence
  Synthetic construct, binding module peptide
RWIPRPPRRACRR

SEQ ID NO: 332 - protein, 13 aa, Artificial Sequence
  Synthetic construct, binding module peptide
SFWPFCPTTWANY

SEQ ID NO: 333 - protein, 13 aa, Artificial Sequence
  Synthetic construct, binding module peptide
SIFQFNPFPEGFF

SEQ ID NO: 334 - protein, 13 aa, Artificial Sequence
  Synthetic construct, binding module peptide
SLFFMPPERLDHR

SEQ ID NO: 335 - protein, 13 aa, Artificial Sequence
  Synthetic construct, binding module peptide
SNRHRRPRRRWRM

SEQ ID NO: 336 - protein, 13 aa, Artificial Sequence
  Synthetic construct, binding module peptide
TFFTNKPFSYHFE

SEQ ID NO: 337 - protein, 13 aa, Artificial Sequence
  Synthetic construct, binding module peptide
TTPVQPPGEVSQV

SEQ ID NO: 338 - protein, 13 aa, Artificial Sequence
  Synthetic construct, binding module peptide
TYNSFFPFRHFAE

SEQ ID NO: 339 - protein, 13 aa, Artificial Sequence
  Synthetic construct, binding module peptide
VKIRRRPRRMRLM

SEQ ID NO: 340 - protein, 13 aa, Artificial Sequence
  Synthetic construct, binding module peptide
WKHPPRPYCWKPL

SEQ ID NO: 341 - protein, 13 aa, Artificial Sequence
  Synthetic construct, binding module peptide
YIYTVYPRNSSWF

SEQ ID NO: 342 - protein, 13 aa, Artificial Sequence
  Synthetic construct, binding module peptide
YQPWGPPPPPLVL

SEQ ID NO: 343 - protein, 13 aa, Artificial Sequence
  Synthetic construct, binding module peptide
ARDYDNNMKYYLD

SEQ ID NO: 344 - protein, 13 aa, Artificial Sequence
  Synthetic construct, binding module peptide
ARINNKNVITFQP

SEQ ID NO: 345 - protein, 13 aa, Artificial Sequence
  Synthetic construct, binding module peptide
ASRSSDNISYSST

SEQ ID NO: 346 - protein, 13 aa, Artificial Sequence
  Synthetic construct, binding module peptide
ASSDAGNYEIAGP

SEQ ID NO: 347 - protein, 13 aa, Artificial Sequence
  Synthetic construct, binding module peptide
ATDDENNEMNVGM

SEQ ID NO: 348 - protein, 13 aa, Artificial Sequence
  Synthetic construct, binding module peptide
CSSFSLNWSLSKS

SEQ ID NO: 349 - protein, 13 aa, Artificial Sequence
  Synthetic construct, binding module peptide
DCDHLFNMEQTLR

SEQ ID NO: 350 - protein, 13 aa, Artificial Sequence
  Synthetic construct, binding module peptide
DCVSSNNHDITRG

SEQ ID NO: 351 - protein, 13 aa, Artificial Sequence
  Synthetic construct, binding module peptide
DDERVINSDYSEY

SEQ ID NO: 352 - protein, 13 aa, Artificial Sequence
  Synthetic construct, binding module peptide
DDKNEDNDIPKTP

SEQ ID NO: 353 - protein, 13 aa, Artificial Sequence
  Synthetic construct, binding module peptide
DDTNDMNNSEEKF

SEQ ID NO: 354 - protein, 13 aa, Artificial Sequence
  Synthetic construct, binding module peptide
DDVQDDNDQPYNT

SEQ ID NO: 355 - protein, 13 aa, Artificial Sequence
  Synthetic construct, binding module peptide
DKGNDQNNSPLWA

SEQ ID NO: 356 - protein, 13 aa, Artificial Sequence
  Synthetic construct, binding module peptide
DLVCNNNCRNLFN

SEQ ID NO: 357 - protein, 13 aa, Artificial Sequence
  Synthetic construct, binding module peptide
DNHDKFNQAIQDW

SEQ ID NO: 358 - protein, 13 aa, Artificial Sequence
  Synthetic construct, binding module peptide
DRCNGDNWCNQGD

SEQ ID NO: 359 - protein, 13 aa, Artificial Sequence
  Synthetic construct, binding module peptide
DSEYLSNKSVNDF

SEQ ID NO: 360 - protein, 13 aa, Artificial Sequence
  Synthetic construct, binding module peptide
DTMTDNNQGDDQW

SEQ ID NO: 361 - protein, 13 aa, Artificial Sequence
  Synthetic construct, binding module peptide
EKNWNYNPVMLAN

SEQ ID NO: 362 - protein, 13 aa, Artificial Sequence
  Synthetic construct, binding module peptide
FFSFLPNSDRFQW

SEQ ID NO: 363 - protein, 13 aa, Artificial Sequence
  Synthetic construct, binding module peptide
FFSYWSNFDASWH

SEQ ID NO: 364 - protein, 13 aa, Artificial Sequence
  Synthetic construct, binding module peptide
FHIDDDNDFDTTS

SEQ ID NO: 365 - protein, 13 aa, Artificial Sequence
  Synthetic construct, binding module peptide
FNNFNDNEHNVNK

SEQ ID NO: 366 - protein, 13 aa, Artificial Sequence
  Synthetic construct, binding module peptide
FYNIVNNIFICCI

SEQ ID NO: 367 - protein, 13 aa, Artificial Sequence
  Synthetic construct, binding module peptide
FYWDRLNVGWGLL

SEQ ID NO: 368 - protein, 13 aa, Artificial Sequence
  Synthetic construct, binding module peptide
GDNHNHNTNTIEP

SEQ ID NO: 369 - protein, 13 aa, Artificial Sequence
  Synthetic construct, binding module peptide
HADQDDNCRGKDD

SEQ ID NO: 370 - protein, 13 aa, Artificial Sequence
  Synthetic construct, binding module peptide
HDWDDWNIEAEDG

SEQ ID NO: 371 - protein, 13 aa, Artificial Sequence
  Synthetic construct, binding module peptide
HGSSDTNGQILFE

SEQ ID NO: 372 - protein, 13 aa, Artificial Sequence
  Synthetic construct, binding module peptide
HNWNHNNNLIDRF

SEQ ID NO: 373 - protein, 13 aa, Artificial Sequence
  Synthetic construct, binding module peptide
ICDDDNNMHLYEP

SEQ ID NO: 374 - protein, 13 aa, Artificial Sequence
  Synthetic construct, binding module peptide
IDDSHLNDQCRDD

SEQ ID NO: 375 - protein, 13 aa, Artificial Sequence
  Synthetic construct, binding module peptide
INCNNNNSLNNNN

SEQ ID NO: 376 - protein, 13 aa, Artificial Sequence
  Synthetic construct, binding module peptide
INNVVYNLHDRNN

SEQ ID NO: 377 - protein, 13 aa, Artificial Sequence
  Synthetic construct, binding module peptide
ISNCNINNGNNDS

SEQ ID NO: 378 - protein, 13 aa, Artificial Sequence
  Synthetic construct, binding module peptide
ISNRQSNTSNGMS

SEQ ID NO: 379 - protein, 13 aa, Artificial Sequence
  Synthetic construct, binding module peptide
KFSSLHNISGPKS

SEQ ID NO: 380 - protein, 13 aa, Artificial Sequence
  Synthetic construct, binding module peptide
KNLNQNNNNHFNN

SEQ ID NO: 381 - protein, 13 aa, Artificial Sequence
  Synthetic construct, binding module peptide
KNRVNKNTNVHCF

SEQ ID NO: 382 - protein, 13 aa, Artificial Sequence
  Synthetic construct, binding module peptide
LSNLNYNPNHHDM

SEQ ID NO: 383 - protein, 13 aa, Artificial Sequence
  Synthetic construct, binding module peptide
MRSSSFNFGSFDQ

SEQ ID NO: 384 - protein, 13 aa, Artificial Sequence
  Synthetic construct, binding module peptide
MSNSSSNSSSSSG

SEQ ID NO: 385 - protein, 13 aa, Artificial Sequence
  Synthetic construct, binding module peptide
MYSNYYNFLQKSW

SEQ ID NO: 386 - protein, 13 aa, Artificial Sequence
  Synthetic construct, binding module peptide
NDRNDHNQHRYDH

SEQ ID NO: 387 - protein, 13 aa, Artificial Sequence
  Synthetic construct, binding module peptide
NEMWNNNNVMNHH

SEQ ID NO: 388 - protein, 13 aa, Artificial Sequence
  Synthetic construct, binding module peptide
NENENDNNMNMEI

SEQ ID NO: 389 - protein, 13 aa, Artificial Sequence
  Synthetic construct, binding module peptide
NNNSNHNDPTNAE

SEQ ID NO: 390 - protein, 13 aa, Artificial Sequence
  Synthetic construct, binding module peptide
NNVLNHNCNMFLN

SEQ ID NO: 391 - protein, 13 aa, Artificial Sequence
  Synthetic construct, binding module peptide
NPTKNRNTHLGGR

SEQ ID NO: 392 - protein, 13 aa, Artificial Sequence
  Synthetic construct, binding module peptide
NREVKNNRQKVFK

SEQ ID NO: 393 - protein, 13 aa, Artificial Sequence
  Synthetic construct, binding module peptide
NRNNHFNNEYEWN

SEQ ID NO: 394 - protein, 13 aa, Artificial Sequence
  Synthetic construct, binding module peptide
NTDLNNNQTVSNR

SEQ ID NO: 395 - protein, 13 aa, Artificial Sequence
  Synthetic construct, binding module peptide
PDDAPHNYCTDPL

SEQ ID NO: 396 - protein, 13 aa, Artificial Sequence
  Synthetic construct, binding module peptide
PKDDRNNTVASCE

SEQ ID NO: 397 - protein, 13 aa, Artificial Sequence
  Synthetic construct, binding module peptide
PVNYANNPERVGH

SEQ ID NO: 398 - protein, 13 aa, Artificial Sequence
  Synthetic construct, binding module peptide
PYNGSNNNNATVP

SEQ ID NO: 399 - protein, 13 aa, Artificial Sequence
  Synthetic construct, binding module peptide
QNSQHNNHHCVLG

SEQ ID NO: 400 - protein, 13 aa, Artificial Sequence
  Synthetic construct, binding module peptide
RSSSSGNSSHHHM

SEQ ID NO: 401 - protein, 13 aa, Artificial Sequence
  Synthetic construct, binding module peptide
SESNSNNPGHNLP

SEQ ID NO: 402 - protein, 13 aa, Artificial Sequence
  Synthetic construct, binding module peptide
SFLNNCNHNKLMS

SEQ ID NO: 403 - protein, 13 aa, Artificial Sequence
  Synthetic construct, binding module peptide
SIFNSSNHTHQSM

SEQ ID NO: 404 - protein, 13 aa, Artificial Sequence
  Synthetic construct, binding module peptide
SNMDSSNAPQSWV

SEQ ID NO: 405 - protein, 13 aa, Artificial Sequence
  Synthetic construct, binding module peptide
SNSWNNNEDKHIL

SEQ ID NO: 406 - protein, 13 aa, Artificial Sequence
  Synthetic construct, binding module peptide
SRSGWSNYFCSRQ

SEQ ID NO: 407 - protein, 13 aa, Artificial Sequence
  Synthetic construct, binding module peptide
SSMLHNNPWSKWS

SEQ ID NO: 408 - protein, 13 aa, Artificial Sequence
  Synthetic construct, binding module peptide
SSNQVINTFEDLQ

SEQ ID NO: 409 - protein, 13 aa, Artificial Sequence
  Synthetic construct, binding module peptide
SSQSMPNGSGKET

SEQ ID NO: 410 - protein, 13 aa, Artificial Sequence
  Synthetic construct, binding module peptide
SVSCSCNTSRGCS

SEQ ID NO: 411 - protein, 13 aa, Artificial Sequence
  Synthetic construct, binding module peptide
SVSSKSNEISFCT

SEQ ID NO: 412 - protein, 13 aa, Artificial Sequence
  Synthetic construct, binding module peptide
TDSGSSNSAKAIC

SEQ ID NO: 413 - protein, 13 aa, Artificial Sequence
  Synthetic construct, binding module peptide
TNWCSSNVGSNTS

SEQ ID NO: 414 - protein, 13 aa, Artificial Sequence
  Synthetic construct, binding module peptide
TSSWSFNGTNGSA

SEQ ID NO: 415 - protein, 13 aa, Artificial Sequence
  Synthetic construct, binding module peptide
VADSFDNANYTLD

SEQ ID NO: 416 - protein, 13 aa, Artificial Sequence
  Synthetic construct, binding module peptide
VDDQYDNWDIRDC

SEQ ID NO: 417 - protein, 13 aa, Artificial Sequence
  Synthetic construct, binding module peptide
YNGNYHNHGLNIR

SEQ ID NO: 418 - protein, 13 aa, Artificial Sequence
  Synthetic construct, binding module peptide
CFVLNCHLVLDRP

SEQ ID NO: 419 - protein, 13 aa, Artificial Sequence
  Synthetic construct, binding module peptide
CRRPFEHALFYAS

SEQ ID NO: 420 - protein, 13 aa, Artificial Sequence
  Synthetic construct, binding module peptide
DSWLLSHSRSKSM

SEQ ID NO: 421 - protein, 13 aa, Artificial Sequence
  Synthetic construct, binding module peptide
DSWWTQHSQAHSD

SEQ ID NO: 422 - protein, 13 aa, Artificial Sequence
  Synthetic construct, binding module peptide
DTNMLNHGMYGHC

SEQ ID NO: 423 - protein, 13 aa, Artificial Sequence
  Synthetic construct, binding module peptide
ENINASHCLSTVD

SEQ ID NO: 424 - protein, 13 aa, Artificial Sequence
  Synthetic construct, binding module peptide
FFSYSGHLVQKVW

SEQ ID NO: 425 - protein, 13 aa, Artificial Sequence
  Synthetic construct, binding module peptide
FMFAVWHDGHIKN

SEQ ID NO: 426 - protein, 13 aa, Artificial Sequence
  Synthetic construct, binding module peptide
FMSQHFHNPMMIR

SEQ ID NO: 427 - protein, 13 aa, Artificial Sequence
  Synthetic construct, binding module peptide
FVFYIMHYCGHFM

SEQ ID NO: 428 - protein, 13 aa, Artificial Sequence
  Synthetic construct, binding module peptide
HFKDDDHMMLYGP

SEQ ID NO: 429 - protein, 13 aa, Artificial Sequence
  Synthetic construct, binding module peptide
HTQHRLHVGQSSS

SEQ ID NO: 430 - protein, 13 aa, Artificial Sequence
  Synthetic construct, binding module peptide
ISNSWYHWSWEMW

SEQ ID NO: 431 - protein, 13 aa, Artificial Sequence
  Synthetic construct, binding module peptide
LCFYEYHFMQCAM

SEQ ID NO: 432 - protein, 13 aa, Artificial Sequence
  Synthetic construct, binding module peptide
LGLSDSHYECSFR

SEQ ID NO: 433 - protein, 13 aa, Artificial Sequence
  Synthetic construct, binding module peptide
LRSTSFHFRCAKC

SEQ ID NO: 434 - protein, 13 aa, Artificial Sequence
  Synthetic construct, binding module peptide
LSVFSHHKWVYTS

SEQ ID NO: 435 - protein, 13 aa, Artificial Sequence
  Synthetic construct, binding module peptide
MAMHHMHHMANNL

SEQ ID NO: 436 - protein, 13 aa, Artificial Sequence
  Synthetic construct, binding module peptide
MSSFDVHRSHTNS

SEQ ID NO: 437 - protein, 13 aa, Artificial Sequence
  Synthetic construct, binding module peptide
PGSLSEHIYQAWS

SEQ ID NO: 438 - protein, 13 aa, Artificial Sequence
  Synthetic construct, binding module peptide
PSSASMHIASSCI

SEQ ID NO: 439 - protein, 13 aa, Artificial Sequence
  Synthetic construct, binding module peptide
QYWWIWHKSDSGS

SEQ ID NO: 440 - protein, 13 aa, Artificial Sequence
  Synthetic construct, binding module peptide
SGQSNSHHDKTIC

SEQ ID NO: 441 - protein, 13 aa, Artificial Sequence
  Synthetic construct, binding module peptide
SGQSVFHHFFPND

SEQ ID NO: 442 - protein, 13 aa, Artificial Sequence
  Synthetic construct, binding module peptide
SHVSLYHASTDSD

SEQ ID NO: 443 - protein, 13 aa, Artificial Sequence
  Synthetic construct, binding module peptide
SMSSSKHMDMDCF

SEQ ID NO: 444 - protein, 13 aa, Artificial Sequence
  Synthetic construct, binding module peptide
SSCLPSHVRSDTK

SEQ ID NO: 445 - protein, 13 aa, Artificial Sequence
  Synthetic construct, binding module peptide
SSGMSEHTPLCSE

SEQ ID NO: 446 - protein, 13 aa, Artificial Sequence
  Synthetic construct, binding module peptide
SSPSFPHMWSEDE

SEQ ID NO: 447 - protein, 13 aa, Artificial Sequence
  Synthetic construct, binding module peptide
VHSESWHSYSIHA

SEQ ID NO: 448 - protein, 13 aa, Artificial Sequence
  Synthetic construct, binding module peptide
VNNAMGHMGMMWC

SEQ ID NO: 449 - protein, 13 aa, Artificial Sequence
  Synthetic construct, binding module peptide
VSCSSRHYSISWS

SEQ ID NO: 450 - protein, 13 aa, Artificial Sequence
  Synthetic construct, binding module peptide
WTWKRQHHRSSLY

SEQ ID NO: 451 - protein, 13 aa, Artificial Sequence
  Synthetic construct, binding module peptide
YISFFEHGQIVDS

SEQ ID NO: 452 - protein, 19 aa, Artificial Sequence
  Synthetic construct, binding module peptide
SCLVFMRPYFLLVFLMCWS

SEQ ID NO: 453 - protein, 19 aa, Artificial Sequence
  Synthetic construct, binding module peptide
SCTFGFPCVMSLVNHVPSS

SEQ ID NO: 454 - protein, 19 aa, Artificial Sequence
  Synthetic construct, binding module peptide
SCLYCLNYANFSDPMTMFS

SEQ ID NO: 455 - protein, 13 aa, Artificial Sequence
  Synthetic construct, binding module peptide
GFAWSSYLGTTVH

SEQ ID NO: 456 - protein, 13 aa, Artificial Sequence
  Synthetic construct, binding module peptide
LFGPIEYTQFLAN

SEQ ID NO: 457 - protein, 13 aa, Artificial Sequence
  Synthetic construct, binding module peptide
FFSFFFPASAWGS

SEQ ID NO: 458 - protein, 20 aa, Artificial Sequence
  Synthetic construct, binding module peptide
FFSFFFPASAWGSSGSSRGD

SEQ ID NO: 459 - protein, 12 aa, Artificial Sequence
  Synthetic construct, binding module peptide
LLSLLLPGSSGK

SEQ ID NO: 460 - protein, 12 aa, Artificial Sequence
  Synthetic construct, binding module peptide
IISIIIPGSSGK

SEQ ID NO: 461 - protein, 12 aa, Artificial Sequence
  Synthetic construct, binding module peptide
FWSFWFPGSSGK

SEQ ID NO: 462 - protein, 33 aa, Artificial Sequence
  Synthetic construct, binding module peptide
SCSDCLKSVDFIPSSLASSSSGRGDSPGRGDSP